(19) United States
(12) Patent Application Publication
(10) Pub. No.: US 2017/0182087 A1
(43) Pub. Date: Jun. 29, 2017

(54) MAIN CHAIN POLYAMINES

(71) Applicant: GENZYME CORPORATION, Cambridge, MA (US)

(72) Inventors: Pradeep K. DHAL, Westford, MA (US); Magnus BESEV, Newton, MA (US)

(73) Assignee: GENZYME CORPORATION, Cambridge, MA (US)

(21) Appl. No.: 15/325,290

(22) PCT Filed: Jul. 10, 2015
(86) PCT No.: PCT/US2015/039881

§ 371 Date: Jan. 10, 2017

Related U.S. Application Data
(60) Provisional application No. 62/023,330, filed on Jul. 11, 2014.

(51) Int. Cl. A61K 31/787; A61K 31/785; C08G 12/06; C08G 12/34; C08G 12/26; C08G 73/06

(52) U.S. Cl. CPC A61K 31/787; C08G 12/26; C08G 73/0627; C08G 12/06; C08G 12/34; A61K 31/785

(57) ABSTRACT

Main chain polyamines comprise amine and ammonium groups along the polymer backbone. Main chain polyamines can be used as pharmaceutical agents and in pharmaceutical compositions. The main chain polyamines are particularly useful in the treatment or prevention of mucositis and infection, specifically oral mucositis and surgical site infection.

### CROSS-REFERENCE TO RELATED APPLICATIONS

This application claims the benefit of priority to U.S. Provisional Patent Application No. 62/023,330, filed Jul. 11, 2014, which is hereby incorporated by reference in its entirety.

### STATEMENT REGARDING FEDERALLY SPONSORED RESEARCH

Not applicable

### THE NAMES OF THE PARTIES TO A JOINT RESEARCH AGREEMENT

Not applicable

### INCORPORATION-BY-REFERENCE OF MATERIAL SUBMITTED ON COMPACT DISC

Not applicable

### BACKGROUND OF THE INVENTION

Field of the Invention

This invention relates to main chain polyamines. Main chain polyamines comprise amine and ammonium groups along the polymer chain. Main chain polyamines can be used as antimicrobial, antiviral and antifungal agents for the treatment of various infections. This invention further relates to the use of main chain polyamines as pharmaceutical agents and in pharmaceutical compositions.

Mucositis is defined as inflammation and/or ulceration of a mucous membrane in the digestive tract. Mucositis can occur in the stomach, intestines and mouth. The disorder is characterized by breakdown of mucosa, which results in redness, swelling and/or the formation of ulcerative lesions.

Oral mucositis is a common dose-limiting toxicity of drug and radiation therapy for cancer; it occurs to some degree in more than one third of all patients receiving anti-neoplastic drug therapy. In granulocytopenic patients, the ulcerations that accompany mucositis are frequent portals of entry for indigenous oral bacteria leading to sepsis or bacteremia. There are about one million occurrences of oral mucositis annually in the United States. Mucositis also includes mucositis that develops spontaneously in a healthy patient not receiving anti-cancer therapy, as in the case of a canker sore or mouth ulcer. Improved therapies to treat mucositis are needed.

Surgical site infection (SSI) is an infection associated with a surgical procedure. Postoperative SSIs are a major source of illness, and less commonly death, in surgical patients (Nichols R L., 2001). The Guideline for Prevention of Surgical Site Infection (1999) sets forth recommendations for preventing SSIs.
Preoperative measures including proper preparation of the patient, antisepsis for surgical team, management of surgical personnel who exhibit signs of transmissible infectious illness, and antimicrobial prophylaxis.
Intra-operative measures including proper ventilation in the operating room, cleaning and disinfecting of surfaces in the surgical environment, microbiologic sampling, sterilization of surgical instruments, proper surgical attire and drapes, and proper asepsis and surgical technique.
Proper incision care post-operation, including sterile dressings and hand washing before and after dressing changes.
Continued surveillance of the surgical wound during the healing process.

Despite these recommendations, SSIs develop in about 1 to 3 of every 100 patients who have surgery (CDC.gov, 2011). These infections can result in major complications that increase the costs and duration of post-operative hospital stays. Accordingly, novel approaches to mitigating SSIs are needed.

Cystic fibrosis (CF) is a genetic disease caused by a mutation in the cystic fibrosis transmembrane conductor regulator (CFTR) protein that results in abnormally thick and sticky mucus (Yu Q, et al., 2012). The thick, sticky mucus of a CF patient leads to compromised mucus clearance and lung infection. Chronic airway infections are one of the most common and debilitating manifestations of CF (Tümmler B and C Kiewitz, 1999). The stagnant mucus becomes a breeding ground for bacteria like Pseudomonas aeruginosa, which causes chronic airway infections (Moreau-Marquis S, G A O'Toole and B A Stanton, 2009). Despite the use of traditional antibacterial therapies in CF patients, most CF patients are afflicted with a chronic P. aeruginosa infection as teenagers and adults, leading to increased morbidity and mortality (Hoiby N, B Frederiksen B, T Pressler, 2005). In chronic P. aeruginosa infection, the P. aeruginosa forms biofilms, resulting in a greater tolerance to antibiotics and increasing difficulty in treatment (Yu Q, et al., 2012). Effective, novel treatments to assuage the effects of bacterial infection and biofilm formation in CF patients are needed.

### Definitions

As used herein, the term “amino” means a functional group having a nitrogen atom and 1 to 2 hydrogen atoms. “Amino” generally may be used herein to describe a primary, secondary, or tertiary amine, and those of skill in the art will readily be able to ascertain the identification of which in view of the context in which this term is used in the present disclosure. The term “amine” or “amine group” or “ammonia group” means a functional group containing a nitrogen atom derived from ammonia (NH₃). The amine groups may be primary amines, meaning the nitrogen is bonded to two hydrogen atoms and one substituent group comprising a substituted or unsubstituted alkyl or aryl group or an aliphatic or aromatic group. The amine groups may be secondary amines meaning, the nitrogen is bonded to one hydrogen atom and two substituent groups comprising a substituted or unsubstituted alkyl or aryl groups or an aliphatic or aromatic group, as defined below. The amine groups may be tertiary amines meaning the nitrogen is bonded to three substituent groups comprising a substituted or unsubstituted alkyl or aryl groups or an aliphatic or aromatic group. The amine groups may also be quaternary amines meaning the designated amine group is bonded to a fourth group, resulting in a positively charged ammonium group.

As used herein, the term “amide group” means a functional group comprising a carbonyl group linked to a nitrogen. A “carbonyl group” means a functional group comprising a carbon atom double bonded to an oxygen atom, represented by (C═O).

The term “alkane” means a saturated hydrocarbon, bonded by single bonds. Alkanes can be linear or branched. “Cycloalkanes” are saturated hydrocarbons rings bonded by single bonds.

As used herein, the term “(C₁-C₁₀)alkyl” means a saturated straight chained or branched or cyclic hydrocarbon consisting essentially of 1 to 10 carbon atoms and a corresponding number of hydrogen atoms. Typically straight chained or branched groups have from one to ten carbons, or more typically one to five carbons. Exemplary (C₁-C₁₀)alkyl groups include methyl (represented by —CH₃), ethyl (represented by —CH₂—CH₃), n-propyl, isopropyl, n-butyl, isobutyl, etc. Other (C₁-C-₁)alkyl groups will be readily apparent to those of skill in the art given the benefit of the present disclosure.

As used herein, the term “(C₂-C₉)heteroalkyl” means a saturated straight chained or branched or cyclic hydrocarbon consisting essentially of 2 to 10 atoms, wherein 2 to 9 of the atoms are carbon and the remaining atom(s) is selected from the group consisting of nitrogen, sulfur, phosphorus and oxygen. Exemplary (C₂-C₉)heteroalkyl groups will be readily apparent to those of skill in the art given the benefit of the present disclosure.

As used herein, the term “(C₃-C₁₀)cycloalkyl” means a nonaromatic saturated hydrocarbon group, forming at least one ring consisting essential of 3 to 10 carbon atoms and a corresponding number of hydrogen atoms. (C₃-C₁₀)cycloalkyl groups can be monocyclic or multicyclic. Individual rings of multicyclic cycloalkyl groups can have different connectivities, for example, fused, bridged, spiro, etc., in addition to covalent bond substitution. Exemplary (C₃-C₁₀)cycloalkyl groups include cyclopropyl, cyclobutyl, cyclopentyl, cyclohexyl, norbornanyl, bicyclo-octanyl, octahydro-pentalenyl, spiro-decanyl, cyclopropyl substituted with cyclobutyl, cyclobutyl substituted with cyclopentyl, cyclohexyl substituted with cyclopropyl, etc. Other (C₃-C₁₀)cycloalkyl groups will be readily apparent to those of skill in the art given the benefit of the present disclosure.

As used herein, the term “(C₂-C₉)heterocycloalkyl” means a nonaromatic group having 3 to 10 atoms that form at least one ring, wherein 2 to 9 of the ring atoms are carbon and the remaining ring atom(s) is selected from the group consisting of nitrogen, sulfur, and oxygen. (C₂-C₉)heterocycloalkyl groups can be monocyclic or multicyclic. Individual rings of such multicyclic heterocycloalkyl groups can have different connectivities, for example, fused, bridged, spiro, etc., in addition to covalent bond substitution. Exemplary (C₂-C₉)heterocycloalkyl groups include pyrrolidinyl, tetrahydrofuranyl, dihydrofuranyl, tetrahydropyranyl, pyranyl, thiopyranyl, aziridinyl, azetidinyl, oxiranyl, methylenedioxyl, chromenyl, barbituryl, isoxazolidinyl, 1,3-oxazolidin-3-yl, isothiazolidinyl, 1,3-thiazolidin-3-yl, 1,2-pyrazolidin-2-yl, 1,3-pyrazolidin-1-yl, piperidinyl, thiomorpholinyl, 1,2-tetrahydrothiazin-2-yl, 1,3-tetrahydrothiazin-3-yl, tetrahydrothiadiazinyl, morpholinyl, 1,2-tetrahydrodiazin-2-yl, 1,3-tetrahydrodiazin-1-yl, tetrahydroazepinyl, piperazinyl, piperizin-2-onyl, piperizin-3-onyl, chromanyl, 2-pyrrolinyl, 3-pyrrolinyl, imidazolidinyl, 2-imidazolidinyl, 1,4-dioxanyl, 8-azabicyclo[3.2.1]octanyl, 3-azabicyclo[3.2.1]octanyl, 3,8-diazabicyclo[3.2.1]octanyl, 2,5-diazabicyclo[2.2.1]heptanyl, 2,5-diazabicyclo[2.2.2]octanyl, octahydro-2H-pyrido[1,2-a]pyrazinyl, 3-azabicyclo[4.1.0]heptanyl, 3-azabicyclo[3.1.0]hexanyl, 2-azaspiro[4.4]nonanyl, 7-oxa-1-aza-spiro[4.4]nonanyl, 7-azabicyclo[2.2.2]heptanyl, octahydro-1H-indolyl, etc. The (C₂-C₉)heterocycloalkyl group is typically attached to the main structure via a carbon atom or a nitrogen atom. Other (C₂-C₉)heterocycloalkyl groups will be readily apparent to those of skill in the art given the benefit of the present disclosure.

The term “aliphatic group” or “aliphatic” means a non-aromatic group consisting of carbon and hydrogen, and may optionally include one or more double and/or triple bonds. An aliphatic group may be straight chained, branched or cyclic and typically contains between about one and about 24 carbon atoms.

The term “aryl group” may be used interchangeably with “aryl,” “aryl ring,” “aromatic,” “aromatic group,” and “aromatic ring.” Aryl groups include carbocyclic aromatic groups, typically with six to fourteen ring carbon atoms. Aryl groups also include heteroaryl groups, which typically have five to fourteen ring atoms with one or more heteroatoms selected from nitrogen, oxygen and sulfur.

As used herein, the term “(C₆-C₁₄)aryl” means an aromatic functional group having 6 to 14 carbon atoms that form at least one ring.

As used herein, the term “(C₂-C₉)heteroaryl” means an aromatic functional group having 5 to 10 atoms that form at least one ring, wherein 2 to 9 of the ring atoms are carbon and the remaining ring atom(s) is selected from the group consisting of nitrogen, sulfur, and oxygen. (C₂-C₉)heteroaryl groups can be monocyclic or multicyclic. Individual rings of such multicyclic heteroaryl groups can have different connectivities, for example, fused, etc., in addition to covalent bond substitution. Exemplary (C₂-C₉)heteroaryl groups include furyl, thienyl, thiazolyl, pyrazolyl, isothiazolyl, oxazolyl, isoxazolyl, pyrrolyl, triazolyl, tetrazolyl, imidazolyl, 1,3,5-oxadiazolyl, 1,2,4-oxadiazolyl, 1,2,3-oxadiazolyl, 1,3,5-thiadiazolyl, 1,2,3-thiadiazolyl, 1,2,4-thiadiazolyl, pyridyl, pyrimidyl, pyrazinyl, pyridazinyl, 1,2,4-triazinyl, 1,2,3-triazinyl, 1,3,5-triazinyl, pyrazolo[3,4-b]pyridinyl, cinnolinyl, pteridinyl, purinyl, 6,7-dihydro-5H-Wpyrindinyl, benzo[b]thiophenyl, 5,6,7,8-tetrahydro-quinolin-3-yl, benzoxazolyl, benzothiazolyl, benzisothiazolyl, benzisoxazolyl, benzimidazolyl, thianaphthenyl, isothianaphthenyl, benzofuranyl, isobenzofuranyl, isoindolyl, indolyl, indolizinyl, indazolyl, isoquinolyl, quinolyl, phthalazinyl, quinoxalinyl, quinazolinyl and benzoxazinyl, etc. The (C₂-C₉)heteroaryl group is typically attached to the main structure via a carbon atom, however, those of skill in the art will realize when certain other atoms, for example, hetero ring atoms, can be attached to the main structure. Other (C₂-C₉)heteroaryl groups will be readily apparent to those of skill in the art given the benefit of the present disclosure.

As used herein, the term “alkyl amine” means an (C₁-C₁₀)alkyl containing a primary, secondary, or tertiary amine group in place of one hydrogen atom, represented by (C₁-C₁₀)alkyl amine and ((C₁-C₁₀)alkyl)₂ amine.

The term “alkyl ester” means a (C₁-C₁₀)alkyl containing an ester group in place of one hydrogen atom, represented by —O(O)C—(C₁-C₁₀)alkyl.

The term “alkyl acid” means an (C₁-C₁₀)alkyl containing a carboxylic acid group in place of one hydrogen atom, represented by (C₁-C₁₀)alkyl-COOH. The term “aliphatic acid” means an acid of nonaromatic hydrocarbons, represented by (C₃-C₁₀)cycloalkyl-COOH.

The term “halo” means a fluorine (F), chlorine (Cl), bromine (Br), iodine (I), or astatine (At) ion.

The term “methoxy” means a (C₁)alkyl containing an oxygen in place of one hydrogen atom, represented by —(O)CH₃.

The term “polyol” means an alcohol containing multiple hydroxyl (—OH) groups.

“Substituted” means the substitution of a carbon in alkyl, heterocyclic or aryl groups with one or more non-carbon substituent. Non-carbon substituents are selected from nitrogen, oxygen, phosphorus and sulfur.

“Unsubstituted” means the group is comprised of only hydrogen and carbon.

The term “polymer” means a molecule comprised of repeating units. The term “repeat unit” or “monomer” means a group in a polymer that repeats or appears multiple times in a polymer. A polymer may be a copolymer if the repeating units or “comonomers” are chemically and structurally different from one another.

The term “pharmaceutically acceptable anion” means an anion that is suitable for pharmaceutical use. Pharmaceutically acceptable anions include but are not limited to halides, carbonate, bicarbonate, sulfate, bisulfate, hydroxide, nitrate, persulfate, sulfite, acetate, ascorbate, benzoate, citrate, dihydrogen citrate, hydrogen citrate, oxalate, succinate, tartrate, taurocholate, glycocholate, and cholate.

The term “pharmaceutically acceptable end group” means an end group that is suitable for pharmaceutical use. Examples of pharmaceutically acceptable end groups include but are not limited to H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl—COOH, —(O)CH₃, —OH, amide, a guanidino group, a guanidinium chloride group, a guanidinobenzene group, a dihydroxy group, and a polyethylene glycol group.

A “guanidino group” is represented by Formula (A):

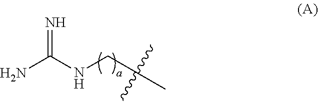

wherein a is an integer from 0 to 25,

A “guanidinium chloride group” is represented by Formula (B),

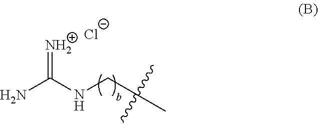

wherein b is an integer from 0 to 25,

A “guanidinobenzene group” is represented by Formula (C),

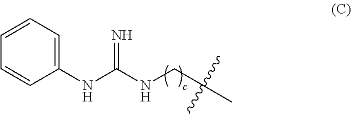

wherein c is an integer from 0 to 25,

A “dihydroxy group” is represented by Formula (D),

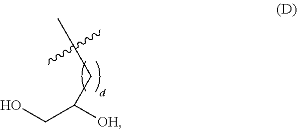

wherein d is an integer from 0 to 25, or

A “polyethylene glycol group” is represented by Formula (E)

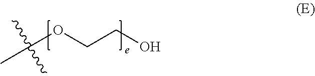

wherein e is an integer from 1 to 400.

The term “effective amount” of a disclosed main chain polyamines is a quantity sufficient to achieve a therapeutic and/or prophylactic effect on the particular condition being treated, such as an amount which results in the prevention or a decrease in the symptoms associated with mucositis, oral mucositis, infection and surgical site infection, and lung infection associated with cystic fibrosis. The precise amount of the disclosed main chain polyamines that is administered will depend on the type and severity of mucositis or infection being treated and on the characteristics of the individual, such as general health, age, sex, body weight and tolerance to drugs.

Related Art

Not applicable

### BRIEF SUMMARY OF THE INVENTION

In a first aspect of the invention, the main chain polyamines are a compound comprising the structure of Formula (I):

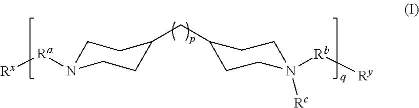

or a pharmaceutically acceptable salt thereof, wherein:
   p is 0, 1, 2, 3, or 4
   q is an integer from 2 to 10,000;
   R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl,
      (C₁-C₁₀)alkyl-COOH, (C₃-C₁₀)cycloalkyl-COOH, —(O)CH₃, —OH, amide.

In another aspect of the invention, the main chain polyamines are a compound comprising the structure of Formula (II):

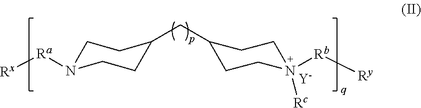

or a pharmaceutically acceptable salt thereof, wherein:
   p is 0, 1, 2, 3, or 4
   q is an integer from 2 to 10,000;
   R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   Y⁻ is a halo or any pharmaceutically acceptable anion.

In another aspect of the invention, the main chain polyamines are a compound comprising the structure of Formula (III):

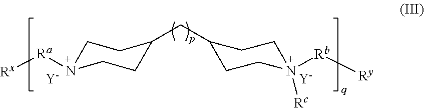

or a pharmaceutically acceptable salt thereof, wherein:
   p is 0, 1, 2, 3, or 4
   q is an integer from 2 to 10,000;
   R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; p2 R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
   Y⁻ is each independently a halo or any pharmaceutically acceptable anion.

In yet another aspect of the invention, the main chain polyamines are a compound comprising the structure of Formula (IV):

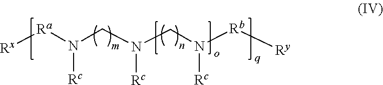

or a pharmaceutically acceptable salt thereof, wherein:
   m is an integer from 0 to 15;
   n is an integer from 0 to 15;
   o is an integer from 0 to 10;
   q is an integer from 2 to 10,000;
   R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, -O(0)C—(C₁-C₁₀)alkyl, (C₁-C₁₀)alkyl-COOH, (C₃-C₁₀)cycloalkyl-COOH, —(O)CH₃, —OH, amide;
   R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide.

In another aspect, the main chain polyamines are a compound comprising the structure of Formula (V):

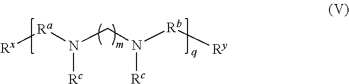

m is an integer from 0 to 15;
q is an integer from 2 to 10,000;
R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{x} and R_{y} are each independently a pharmaceutically acceptable end group or are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide.

In one embodiment of the invention, the main chain polyamines are a compound comprising the structure of Formula (VI):

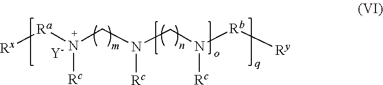

or a pharmaceutically acceptable salt thereof, wherein:
   m is an integer from 0 to 15;
   n is an integer from 0 to 15;
   o is an integer from 0 to 10;
   q is an integer from 2 to 10,000;
   R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
   Y⁻ is a halo or any pharmaceutically acceptable anion.

In another embodiment of the invention, the main chain polyamines are a compound comprising the structure of Formula (VII):

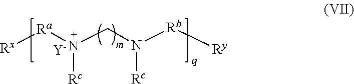

m is an integer from 0 to 15;
q is an integer from 2 to 10,000;
R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, -O(0)C—(C₁-C₁₀)alkyl, (C₁-C₁₀)alkyl-COOH, (C₃-C₁₀)cycloalkyl-COOH, —(O)CH₃, —OH, amide;
R^{x} and R^{y} are each independently a pharmaceutically acceptable end group or are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
Y⁻ is a halo or any pharmaceutically acceptable anion.

In yet another embodiment of the invention, the main chain polyamine is a compound comprising the structure of Formula (VIII):

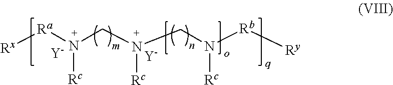

or a pharmaceutically acceptable salt thereof, wherein:
   m is an integer from 0 to 15;
   n is an integer from 0 to 15;
   o is an integer from 0 to 10;
   q is an integer from 2 to 10,000;
   R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   R^{x} and R^{y} are each independently a pharmaceutically acceptable end group or are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
Y⁻is each independently a halo or any pharmaceutically acceptable anion.

In another embodiment, the main chain polyamines are a compound comprising the structure of Formula (IX):

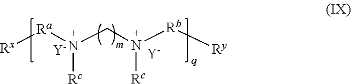

m is an integer from 0 to 15;
q is an integer from 2 to 10,000;
R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, -O(0)C—(C₁-C₁₀)alkyl, (C₁-C₁₀)alkyl-COOH, (C₃-C₁₀)cycloalkyl-COOH, —(O)CH₃, —OH, amide;
R^{x} and R^{y} are each independently a pharmaceutically acceptable end group or are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
Y⁻ is each independently a halo or any pharmaceutically acceptable anion.

In yet another embodiment, the main chain polyamines are a compound comprising the structure of Formula (X):

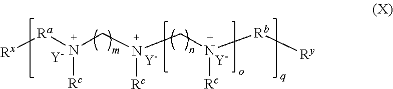

or a pharmaceutically acceptable salt thereof, wherein:
   m is an integer from 0 to 15;
   n is an integer from 0 to 15;
   o is an integer from 0 to 10;
   q is an integer from 2 to 10,000;
   R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
R^{x} and R^{y} are each independently a pharmaceutically acceptable end group or are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
Y⁻ is each independently a halo or any pharmaceutically acceptable anion.

In another embodiment, the main chain polyamines are a compound comprising the structure of Formula (XI):

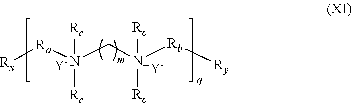

or a pharmaceutically acceptable salt thereof, wherein:
   m is an integer from 0 to 15;
   q is an integer from 2 to 10,000;
   R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
   Y⁻ is each independently a halo or any pharmaceutically acceptable anion.

In another embodiment, the main chain polyamines are a compound comprising the structure of Formula (XII):

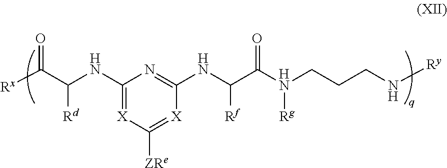

or a pharmaceutically acceptable salt thereof, wherein:
   q is an integer from 2 to 10,000;
   X is each independently N or P;
   Z is NH, O, or S;
   R^{d} and R^{e} are each independently H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{f} and R^{g} are each independently H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide, or R^{f} and R^{g} are taken together with the atoms to which they are attached to form a 4 to 10 member ring,
      wherein the 4 to 10 member ring is optionally substituted by one to three groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, (C₁-C₁₀)alkyl—C(O)O—, COOH—(C₁-C₁₀)alkyl, COOH—(C₃-C₁₀)cycloalkyl, (C₁-C₁₀)alkyl—O—, —OH, —NH₂;
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl,

(C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide.

In another embodiment, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI) or Formula (XII).

In another aspect, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI) or Formula (XII) for use in the treatment of mucositis. In another aspect, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI) or Formula (XII) for use in the treatment of oral mucositis.

In another aspect, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI) or Formula (XII) for use in the treatment of an infection. In another aspect, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI) or Formula (XII) for use in the treatment of a surgical site infection.

In yet another aspect, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI), or Formula (XII) for use in the treatment of a lung infection associated with cystic fibrosis. The invention further relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI), or Formula (XII) for use in the treatment of a lung infection associated with cystic fibrosis, wherein the infection is a Pseudomonas aeruginosa lung infection. The invention further relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI), or Formula (XII) for use in the treatment of a Pseudomonas aeruginosa lung infection, wherein biofilms are present in the Pseudomonas aeruginosa lung.

In another aspect, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI), or Formula (XII) for use in the prevention of mucositis. In another aspect, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI), or Formula (XII) for use in the prevention of oral mucositis.

In another aspect, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI), or Formula (XII) for use in the prevention of an infection. In another aspect, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI), or Formula (XII) for use in the prevention of a surgical site infection.

In yet another aspect, the invention relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI), or Formula (XII) for use in the prevention of a lung infection associated with cystic fibrosis. The invention further relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI), or Formula (XII) for use in the prevention of a lung infection associated with cystic fibrosis, wherein the infection is a Pseudomonas aeruginosa lung infection. The invention further relates to pharmaceutical compositions comprising a compound according to Formula (I), Formula (II), Formula (III), Formula (IV), Formula (V), Formula (VI), Formula (VII), Formula (VIII), Formula (IX), Formula (X), Formula (XI), or Formula (XII) for use in the prevention of a Pseudomonas aeruginosa lung infection, wherein biofilms are present in the Pseudomonas aeruginosa lung.

In another aspect, the invention relates to a method of treating a condition selected from mucositis, oral mucositis, and infection, comprising administering a compound according to Formula (I), Formula (II), Formula (III), Formula (IV), Formula (V), Formula (VI), Formula (VII), Formula (VIII), Formula (IX), Formula (X), Formula (XI), or Formula (XII). In yet another aspect, the invention relates to a method of preventing a condition selected from mucositis, oral mucositis, and infection, comprising administering a compound according to Formula (I), Formula (II), Formula (III), Formula (IV), Formula (V), Formula (VI), Formula (VII), Formula (VIII), Formula (IX), Formula (X), Formula (XI), or Formula (XII).

### BRIEF DESCRIPTION OF THE SEVERAL VIEWS OF THE DRAWING(S)

Not applicable

### DETAILED DESCRIPTION OF THE INVENTION

This invention relates to novel main chain polyamines. The main chain polyamines are polymers or copolymers of varying structures and comprise amine and ammonium groups along the polymer backbone.

The main chain polyamines contain repeat units of amine groups; the amine groups can be secondary, tertiary, and quaternary ammonium groups. The amine groups can be aliphatic or aromatic.

Further, the main chain polyamines of the present invention are of varying molecular weights.

The main chain polyamines are water soluble.

This invention relates to pharmaceutical compositions comprising polymers or copolymers of main chain polyamines. This invention relates to use of main chain polyamines as antimicrobial, antiviral and antifungal agents. This invention also relates to methods of treating mucositis and infection with main chain polyamines. The main chain polyamines and the pharmaceutical compositions comprising polymers or copolymers of main chain polyamines can be administered in multiple dosage forms and for systemic or local administration.

This invention relates to the use of main chain polyamines and pharmaceutical compositions comprising polymers or copolymers of main chain polyamines as anti-infective agents. The main chain polyamines and pharmaceutical compositions comprising polymers or copolymers of main chain polyamines can be used for the treatment of bacterial, fungal, and viral infections, including mucositis, infections and, specifically, surgical site infections.

The main chain polyamines can also be used to coat surfaces of various biomedical devices and other surfaces to prevent infection.

### Specific Embodiments

One embodiment of the present invention is a main chain polyamine polymer or copolymer comprising the structure of Formula (I):

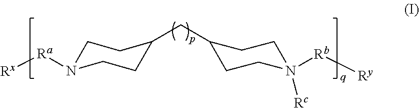

or a pharmaceutically acceptable salt thereof, wherein:
   p is 0, 1, 2, 3, or 4
   q is an integer from 2 to 10,000;
   R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, -(0)CH₃, —OH, amide.

Another embodiment of the present invention is a main chain polyamine polymer or copolymer comprising the structure of Formula (II):

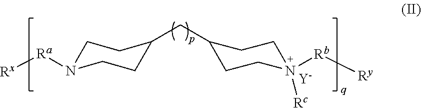

or a pharmaceutically acceptable salt thereof, wherein:
   p is 0, 1, 2, 3, or 4
   q is an integer from 2 to 10,000;
   R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   Y⁻ is a halo or any pharmaceutically acceptable anion.

Another embodiment of the present invention is a main chain polyamine polymer or copolymer comprising the structure of Formula

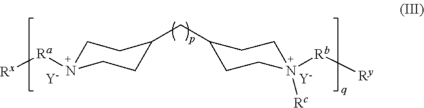

or a pharmaceutically acceptable salt thereof, wherein:
   p is 0, 1, 2, 3, or 4
   q is an integer from 2 to 10,000;
   R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{x} and R_{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
   Y⁻ is each independently a halo or any pharmaceutically acceptable anion.

Another embodiment of the present invention is a main chain polyamine polymer or copolymer comprising g the structure of Formula (IV):

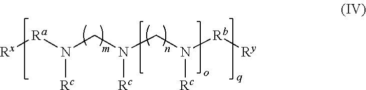

or a pharmaceutically acceptable salt thereof, wherein:
   m is an integer from 0 to 15;
   n is an integer from 0 to 15;
   o is an integer from 0 to 10;
   q is an integer from 2 to 10,000;
   R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   Rₓ and R_{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide.

Yet another embodiment of the present invention is a main chain polyamine polymer or copolymer comprising the structure of Formula (V):

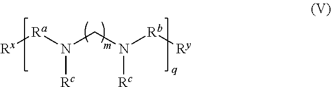

m is an integer from 0 to 15;
q is an integer from 2 to 10,000;
R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
Rₓ and R_{y} are each independently a pharmaceutically acceptable end group or are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide.

One embodiment of the present invention is a main chain polyamine polymer or copolymer comprising the structure of Formula (VI):

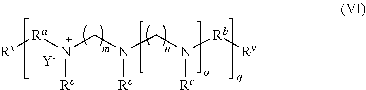

or a pharmaceutically acceptable salt thereof, wherein:
   m is an integer from 0 to 15;
   n is an integer from 0 to 15;
   o is an integer from 0 to 10;
   q is an integer from 2 to 10,000;
   R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   R^{x} and R_{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
   Y⁻ is a halo or any pharmaceutically acceptable anion.

Another embodiment of the present invention is a main chain polyamine polymer or copolymer comprising the structure of Formula (VII):

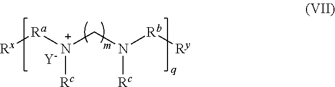

m is an integer from 0 to 15;
q is an integer from 2 to 10,000;
R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, -—(O)CH₃, —OH, amide;
R^{x} and R_{y} are each independently a pharmaceutically acceptable end group or are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
Y⁻ is a halo or any pharmaceutically acceptable anion.

In yet another embodiment of the present invention, the main chain polyamine polymer or copolymer comprises the structure of Formula (VIII):

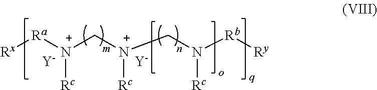

or a pharmaceutically acceptable salt thereof, wherein:
   m is an integer from 0 to 15;
   n is an integer from 0 to 15;
   o is an integer from 0 to 10;
   q is an integer from 2 to 10,000;
   R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
   Y⁻ is each independently a halo or any pharmaceutically acceptable anion.

In still another embodiment of the present invention, main chain polyamine polymer or copolymer comprises the structure of Formula (IX):

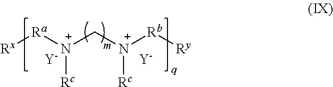

m is an integer from 0 to 15;
q is an integer from 2 to 10,000;
R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{x} and R^{y} are each independently a pharmaceutically acceptable end group or are taken together with the carbons to which they are attached to form a 3 to 10 member ring,
   wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
Y⁻ is each independently a halo or any pharmaceutically acceptable anion.

In another embodiment, the main chain polyamines are a compound comprising the structure of Formula (XI):

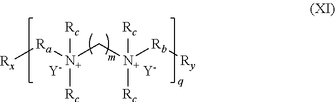

or a pharmaceutically acceptable salt thereof, wherein:
   m is an integer from 0 to 15;
   q is an integer from 2 to 10,000;
   R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   Y⁻ is each independently a halo or any pharmaceutically acceptable anion.

In still another embodiment of the present invention, the main chain polyamine polymer or copolymer comprises the structure of Formula (X):

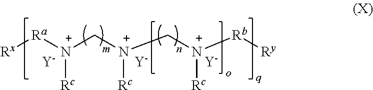

or a pharmaceutically acceptable salt thereof, wherein:
   m is an integer from 0 to 15;
   n is an integer from 0 to 15;
   o is an integer from 0 to 10;
   q is an integer from 2 to 10,000;
   R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
   R^{x} and R^{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COON, —(O)CH₃, —OH, amide; and
   Y⁻ is each independently a halo or any pharmaceutically acceptable anion.

In another embodiment, the main chain polyamine polymer or copolymer comprises the structure of Formula (XII):

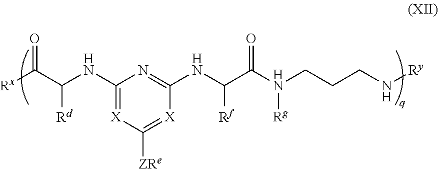

or a pharmaceutically acceptable salt thereof, wherein:
   q is an integer from 2 to 10,000;
   X is each independently N or P;
   Z is NH, O, or S;
   R^{d} and R^{e} are each independently H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
   R^{f} and R_{g} are each independently H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide, or R^{f} and R_{g} are taken together with the atoms to which they are attached to form a 4 to 10 member ring,
      wherein the 4 to 10 member ring is optionally substituted by one to three groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, (C₁-C₁₀)alkyl-C(O)O—, COOH-(C₁-C₁₀)alkyl, COOH-(C₃-C₁₀)cycloalkyl, (C₁-C₁₀)alkyl—O—, —OH, —NH₂;
   R^{x} and R_{y} are each independently a pharmaceutically acceptable end group or are taken together with the carbons to which they are attached to form a 3 to 10 member ring,
      wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide.

In one embodiment of the invention, the main chain polyamines are polymers. In some embodiments, the polymers may comprise a monomer comprising a compound having a repeat unit according to any of Formula (I), Formula (II), Formula (III), Formula (IV), Formula (V), Formula (VI), Formula (VII), Formula (VIII), Formula (IX), Formula (X), Formula (XI), or Formula (XII).

In one embodiment of the invention, the main chain polyamines are copolymers. In some embodiments, the copolymers may comprise a monomer comprising a compound having at least one unit according to any of Formula (I), Formula (II), Formula (III), Formula (IV), Formula (V), Formula (VI), Formula (VII), Formula (VIII), Formula (IX), Formula (X), Formula (XI), or Formula (XII) which is copolymerized with one or more other comonomers or oligomers or other polymerizable groups. Non-limiting examples of suitable comonomers which may be used alone or in combination with at least one unit according to any of Formula (I), Formula (II), Formula (III), Formula (IV), Formula (V), Formula (VI), Formula (VII), Formula (VIII), Formula (IX), Formula (X), Formula (XI), or Formula (XII) to form the main chain polyamines presented in Table 1 or Table 2.

In one aspect of the invention, the main chain polyamines are polymers or copolymers comprised of 2 to 10,000 repeat units according to any of Formula (I), Formula (II), Formula (III), Formula (IV), Formula (V), Formula (VI), Formula (VII), Formula (VIII), Formula (IX), Formula (X), Formula (XI), or Formula (XII). In some embodiments, the main chain polyamines are polymers or copolymers comprised of 2 to 50 repeat units according to any of Formula (I), Formula (II), Formula (III), Formula (IV), Formula (V), Formula (VI), Formula (VII), Formula (VIII), Formula (IX), Formula (X), Formula (XI), or Formula (XII). In an additional embodiment, the main chain polyamines are polymers or copolymers comprised of about 2 to 25 repeat units according to any of Formula (I), Formula (II), Formula (III), Formula (IV), Formula (V), Formula (VI), Formula (VII), Formula (VIII), Formula (IX), Formula (X), Formula (XI), or Formula (XII). In other embodiments, the main chain polyamines are polymers or copolymers comprised of 2 to 40 repeat units according to any of Formula (I), Formula (II), Formula (III), Formula (IV), Formula (V), Formula (VI), Formula (VII), Formula (VIII), Formula (IX), Formula (X), Formula (XI), or Formula (XII). In another embodiment, the main chain polyamines are polymers or copolymers comprised of 5 to 30 repeat units according to any of Formula (I), Formula (II), Formula (III), Formula (IV), Formula (V), Formula (VI), Formula (VII), Formula (VIII), Formula (IX), Formula (X), Formula (XI), or Formula (XII). In yet another embodiment, the main chain polyamines are polymers or copolymers comprised of 5 to 25 repeat units according to any of Formula (I), Formula (II), Formula (III), Formula (IV), Formula (V), Formula (VI), Formula (VII), Formula (VIII), Formula (IX), Formula (X), Formula (XI), or Formula (XII).

In one aspect of the invention, the main chain polyamines have a molecular weight less than about 25,000 g/mol. In another aspect of the invention, the main chain polyamines have a molecular weight less than about 10,000 g/mol. In an additional aspect of the invention, the main chain polyamines have a molecular weight less than about 9,000 g/mol. In yet another aspect of the invention, the main chain polyamines have a molecular weight less than about 5,000 g/mol. In yet another aspect of the invention, the main chain polyamines have a molecular weight less than about 3,000 g/mol. In yet another aspect of the invention, the main chain polyamines have a molecular weight from about 10,000 g/mol to about 3,000 g/mol.

In one aspect of the invention, the main chain polyamines are optionally, independently terminated (Rₓ and R_{y}) with a pharmaceutically acceptable end group. The main chain polyamines according to any of Formula (I), Formula (II), Formula (III), Formula (IV), Formula (V), Formula (VI), Formula (VII), Formula (VIII), Formula (IX), Formula (X),Formula (XI), or Formula (XII) are may be terminated with end groups (Rₓ and R_{y}) that include but are not limited to H, (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, —O(O)C—(C₁-C₁₀)alkyl, (C₁-C₁₀)alkyl-COOH, (C₃-C₁₀)cycloalkyl-COOH, —(O)CH₃, —OH, amide, a guanidino group, a guanidinium chloride group, a guanidinobenzene group, a dihydroxy group, and a polyethylene glycol group.

In one embodiment of the invention, the number of repeat units and the molecular weight are controlled by the synthesis of the main chain polyamine. Methods of preparing preferred main chain polyamines of the invention and controlling for the number of repeat units and molecular are described in Example 3.

TABLE 1
Main Chain Polyamines

| Polymer Description | Structure |
|---|---|
| Poly(1,3-bis(1-pentyl- piperidin-4-yl)propane | 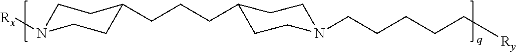 |
| Poly((1,1′-dipentyl)- 4,4-dipiperidine) <10 KDa/>3 KDa Poly((1,1′-dipentyl)- 4,4-dipiperidine) | 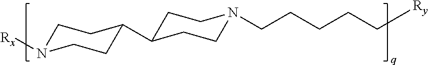 |
| Mw = 8034 (1:1) | |
| Poly((1,1′-dipentyl)- | |
| 4,4-dipiperidine) | |
| Mw = 8710 (1:1) | |
| Poly(1,3-bis(1,4- cyclohexyl)piperidine- 4-yl)propane) Mw = 3116 | 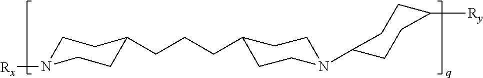 |
| Poly(1,3-bis(1,6- dimethylenepyridine) piperidine-4-yl) propane) | 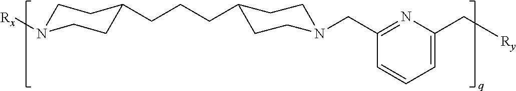 |
| 4,4′-Trimethylene- dipiperidine/ dibutylether heptamere | 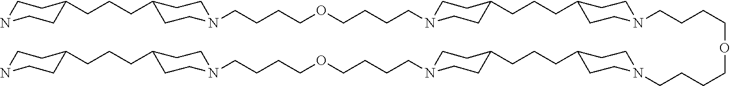 [SMILES: C(CCN1CCC(CCCC2CCNCC2)CC1)COCCCCN1CCC(CCCC2CCN(CCCCOCCCCN3CCC(CCCC4CCN(CCCCOCCCCN5CCC(CCCC6CCNCC6)CC5)CC4)CC3)CC2)CC1] |
| Poly(1,3-bis(1-pentyl- piperidin-4-yl)propane >10 KDa Poly(1,3-bis(1-pentyl- | 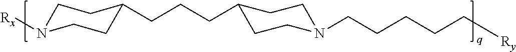 |
| piperidin-4-yl)propane | |
| Mw = <902 | |
| Poly(1,3-bis(1-pentyl- | |
| piperidin-4-yl)propane | |
| Mw = 20667 | |
| Poly(1,3-bis(1-pentyl- | |
| piperidin-4-yl)propane | |
| Mw = 3773 | |
| Poly(1,3-bis(1-pentyl- | |
| piperidin-4-yl)propane | |
| Mw = 2676 | |
| Poly(1,3-bis(1-pentyl- | |
| piperidin-4-yl)propane | |
| Mw = 2159 | |
| Poly(1,3-bis N- (dimethylene) piperidin-4-yl)propane) >10 KDa | 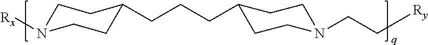 |
| Poly(1,3-bis(1,5- dimethylbenzene) piperidin-4-yl)propane) >10 KDa | 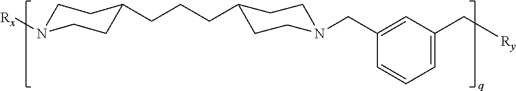 |
| Poly(1,3-bis N-(1,4- dimethybenzene) piperidin-4-yl)propane) >10 KDa Pyridine end capped poly(4,4′- | 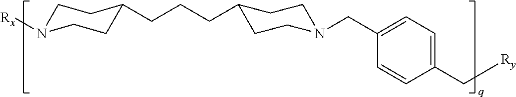 |
| propane-13-diylbis[1-4(- | |
| butoxybutyl)piperidine]) | |
| <3 KDa/>1 KDa | |
| Pyridine end capped poly(4,4′-propane-13- diylbis[1-4(- butoxybutyl) piperidine]) | 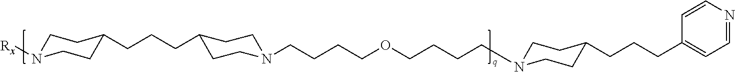 |
| <3 KDa/>1 KDa | |
| Piperidine end capped poly(4,4′-propane- 1,3-diylbis[1-(4- | 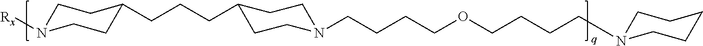 |
| butoxybutyl) | |
| piperidine]) | |
| <3 KDa/>1 KDa | |
| Glycidol (1:1) modifiedPoly (Trimethylene- dipiperidine/ glutaraldehyde/ piperidine) >10 KDa; 1:0.8:0.2 <10 KDa/>3 KDa; 1:0.8:0.2 | 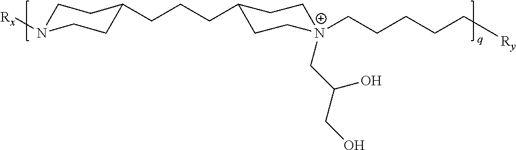 |
| Piperidine end capped poly(1,1′- dipentyl)-4,4- dipiperidine) | 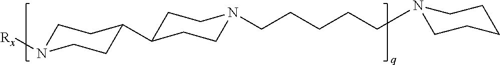 |
| Mw = 3579; 1:0.8:0.2 | |
| Piperidine end capped | |
| poly(1,1′-dipentyl)- | |
| 4,4-dipiperidine) | |
| Mw = 6954; 1:0.8:0.2 | |
| Piperidine end capped | |
| poly(1,1′-dipentyl)- | |
| 4,4-dipiperidine) | |
| <10 KDa/>3 KDa; | |
| 1:0.8:0.2 | |
| Poly(Bispiperidine/ glutaraldehyde/ PEG12 piperidine) Mw = 7757; 0.6:1:0.4 Poly(Bispiperidine/ glutaraldehyde/ | 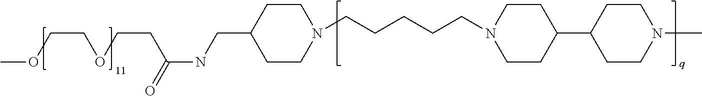 |
| PEG12 piperidine) | |
| Mw = 4213; 0.8:1:0.2 | |
| Poly(Bispiperidine/ | |
| glutaraldehyde/ | |
| PEG12 piperidine) | |
| 0.8:1:0.2 | |
| Poly(Bispiperidine/ | |
| glutaraldehyde/ | |
| PEG12 piperidine) | |
| Mw = 8423; 0.8:1:0.4 | |
| Poly(Bispiperidine/ | |
| glutaraldehyde/ | |
| PEG12 piperidine) | |
| Mw = 3301; 0.8:1:0.4 | |
| Poly(Bispiperidine/ | |
| glutaraldehyde/ | |
| PEG12 piperidine) | |
| <10 KDa/>3 KDa; | |
| 0.6:1:0.4 | |
| Poly(Bispiperidine/ | |
| glutaraldehyde/ | |
| PEG12 piperidine); | |
| >10 KDa; 0.8:1:0.2 | |
| Poly(4,4′-Dipiperidine/ Glutaraldehyde/ Piperidine-PEG4 MW = 8322, 1:0.8:0.2 Poly(4,4′-Dipiperidine/ Glutaraldehyde/ | 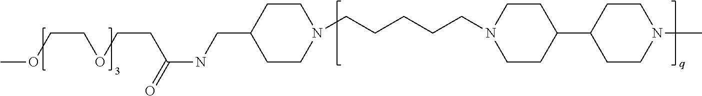 |
| Piperidine-PEG4 | |
| MW = 3791, 1:0.8:0.2 | |
| Poly(4,4′-Dipiperidine/ | |
| Glutaraldehyde/ | |
| Piperidine-PEG4 | |
| MW = 4368, 1:0.8:0.4 | |
| Poly(4,4′-Dipiperidine/ | |
| Glutaraldehyde/ | |
| Piperidine-PEG4 | |
| MW = 4488, 1:0.8:0.4 | |
| Poly(4,4′-Dipiperidine/ | |
| Glutaraldehyde/ | |
| Piperidine-PEG4 | |
| MW <10 KDa/>3 KDa | |
| fraction; 1:0.8:0.4 | |
| Spermidin(N4-butanoic acid amide)/ glyoxal polymer Mw = 3002; 1:1 Spermidin(N4-butanoic acid amide)/ | 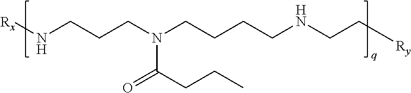 |
| glyoxal polymer | |
| Mw = <963; 1:1 | |
| Spermidine(N4- hexanoic acid amide)/glyoxal polymer <10K | 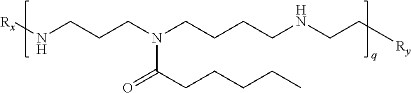 |
| Spermidine(N4- propionic acid amide)/glyoxal polymer >10K | 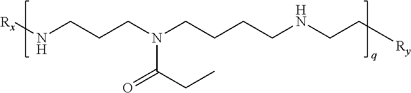 |
| Piperidine end capped poly(N1,N6-dimethyl- N1,N6-dipentyl- hexane-1,6-diamine) <3 KDa/>1 KDa; 1:0.8:0.2 | 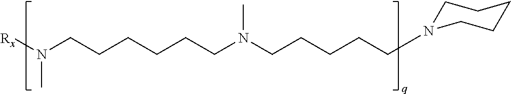 |
| Piperidine end capped poly(N1,N6-dimethyl- N1,N6-diethyl- hexane-1,6-diamine) <3 KDa/>1 KDa; 1:0.8:0.2 | 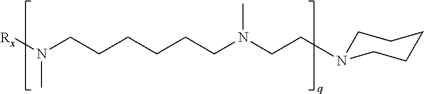 |
| Piperidine end capped poly(N1,N6-dimethyl- N1,N6-dibutyl- hexane-1,6-diamine) <3 KDa/>1 KDa; 1:0.8:0.2 | 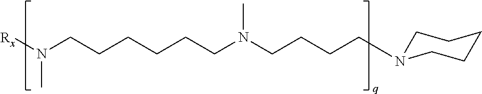 |
| Piperidine end capped poly(1,3-bis(1-pentyl- piperidin-4-yl)propane | 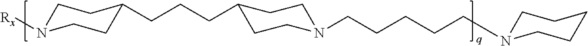 |
| <10 KDa/>3 KDa; | |
| 1:0.8:0.2 | |
| Piperidine end capped | |
| poly(1,3-bis(1-pentyl- | |
| piperidin-4-yl)propane | |
| <3 KDa/>1 KDa; | |
| 1:0.8:0.2 | |
| Piperidine end capped poly(1,3-bis(1-butyl- piperidin-4-yl)propane <10 KDa/>3 KDa; 1:0.8:0.2 | 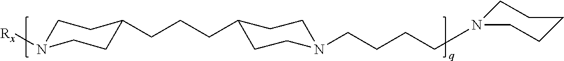 |
| Piperidine end capped poly(N1,N6-dibenzyl- N1,N6-dipentyl- hexane-1,6-diamine) | 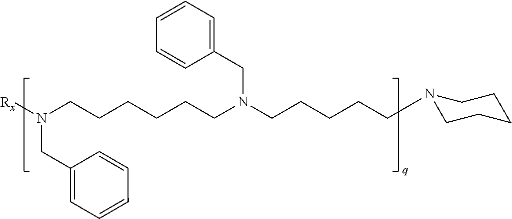 |
| Piperidine end capped poly(N1,N6-dipentyl- hexane-1,6-diamine) | 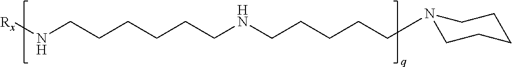 |
| 4-(tetraethyleneglycol amidomethyl)- piperidine end capped poly(1,3-bis(1- pentylpiperidin-4- | 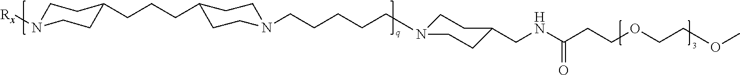 |
| yl)propane) | |
| <10 KDa/>3 KDa; | |
| 1:0.8:0.2 | |
| 4-(dodecaethylene- glycol amidomethyl)- piperidine end capped poly(1,3-bis(1- pentylpiperidin-4- | 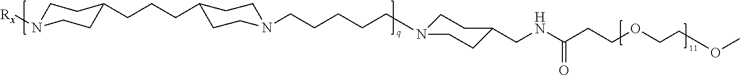 |
| yl)propane) | |
| Poly{N1-ethylene- N1,N1,N6,N6- tetramethyl-N6- propylenehexane- 1,6-diaminium chloride} | 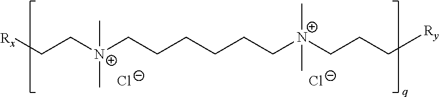 |
| Poly(6-((3-(4-(3- aminopropyl) piperazin-1-yl)propyl) amino)-6-oxohexanoic acid) <10 KDa/>3 KDa; 1.24:1 | 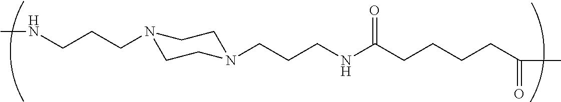 |
| Poly(5-((3-(4-(3-(1-(3- aminopropyl)piperidin-4- yl)propyl)piperidin-1- yl)propyl)amino)-5- oxopentanoic acid) <10 KDa/>3 KDa; 1.2:1 | 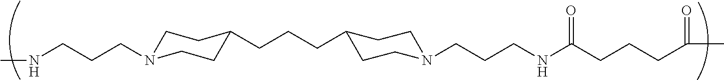 |
| Poly(5-((3-(4-(3-(1-(3- | |
| aminopropyl)piperidin- | |
| 4-yl)propyl)piperidin-1- | |
| yl)propyl)amino)-5- | |
| oxopentanoic acid) | |
| >10 KDa; 1.2:1 | |
| Po1y(6-((3-(4-(3-(1-(3- aminopropyl)piperidin-4- yl)propyl)piperidin-1- yl)propyl)amino)-6- oxohexanoic acid) <10 KDa/>3 KDa; 1.2:1 Poly(6-((3-(4-(3-(1-(3- | 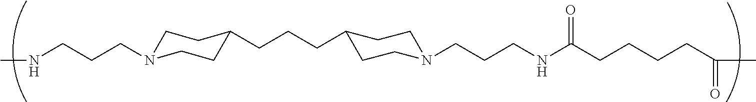 |
| aminopropyl)piperidin-4- | |
| yl)propyl)piperidin-1- | |
| yl)propyl)amino)-6- | |
| oxohexanoic acid) | |
| >10 KDa; 1.2:1 | |
| Poly(8-((3-(4-(3-(1-(3- aminopropyl)piperidin-4- yl)propyl)piperidin-1- yl)propyl)amino)-8- oxooctanoic acid) <10 KDa/>3 KDa; 1.2:1 | 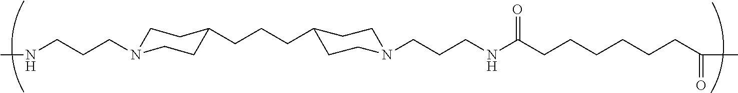 |
| Poly(8-((3-(4-(3-(1-(3- | |
| aminopropyl)piperidin-4- | |
| yl)propyl)piperidin-1- | |
| yl)propyl)amino)-8- | |
| oxooctanoic acid) | |
| >10 KDa; 1.2:1 | |
| Poly(1-(3-(4-(3-(1-(3- aminopropyl)piperidin-4- yl)propyl)piperidin-1- yl)propyl)-3- (4-ureidobutyl)urea) <10 KDa/>3 KDa; 1.2:1 | 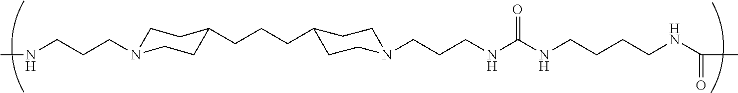 |
| Poly(1-(3-(4-(3-(1-(3- | |
| aminopropyl)piperidin-4- | |
| yl)propyl)piperidin-1- | |
| yl)propyl)-3- | |
| (4-ureidobutyl)urea) | |
| >10 KDa; 1.2:1 | |
| Poly(1-(2-((6- aminohexyl)amino)-2- oxoethyl)-4- (carboxymethyl)-1,4- diazabicyclo[2.2.2] octane-1,4-diium bromide) | 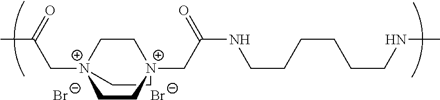 |
| 1:1 diester/diamine | |
| Poly(1-(2-((6- | |
| aminohexyl)amino)-2- | |
| oxoethyl)-4- | |
| (carboxymethyl)-1,4- | |
| diazabicyclo[2.2.2] | |
| octane-1,4-diium | |
| bromide) | |
| 1:1 diester/diamine | |
| Poly(1-(2-((6- | |
| aminohexyl)amino)-2- | |
| oxoethyl)-4- | |
| (carboxymethyl)-1,4- | |
| diazabicyclo[2.2.2] | |
| octane-1,4-diium | |
| bromide) | |
| 1:2 diester/diamine | |
| Poly(1-(2-((8- aminooctyl)amino)-2- oxoethyl)-4- 4-(carboxymethyl)-1,4- diazabicyclo[2.2.2] octane-1,4- diium bromide) | 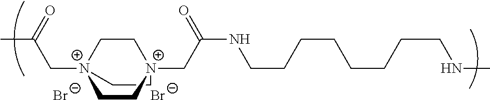 |
| 1:1 diester/diamine | |
| Poly(1-(2-((8- | |
| aminooctyl)amino)-2- | |
| oxoethyl)-4- | |
| (carboxymethyl)-1,4- | |
| diazabicyclo[2.2.2] | |
| octane-1,4-diium | |
| bromide) | |
| 1:2 diester/diamine | |
| Poly(trimethylene dipiperidine- co-piperidine-co-2,3- Butanedione) >10 KDa Poly(trimethylene | 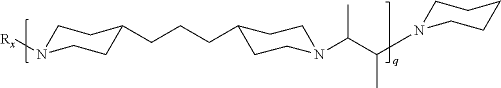 |
| dipiperidine- | |
| co-piperidine-co-2,3- | |
| Butanedione) | |
| <10 KDa | |
| Poly(dipiperidine-co- piperidine-co-2,3- Butanedione) >10 KDa Poly(dipiperidine-co- piperidine-co-2,3- | 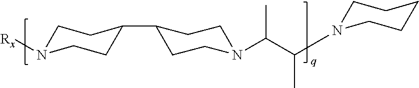 |
| Butanedione) | |
| <10 KDa | |
| Poly(trimethylene dipiperidine:piperidine- co-2,2-Dimethyl- 3,5-Hexanedione) | 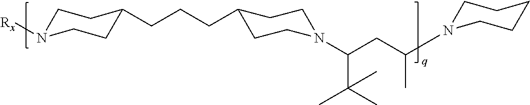 |
| Poly(trimethylene dipiperidine:piperidine- co-(±)- Camphorquinone) | 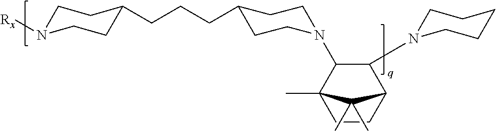 |
| Poly((4-((2- aminoethyl)thio)-6- ((2-((3-aminopropyl) amino)-2-oxoethyl) amino)-1,3,-triazin-2- yl)glycine) >10 KDa Poly((4-((2- aminoethyl)thio)-6- ((2-((3-aminopropyl) amino)-2-oxoethyl) amino)-1,3,5-triazin-2- yl)glycine) <3 KDa/>1 KDa | 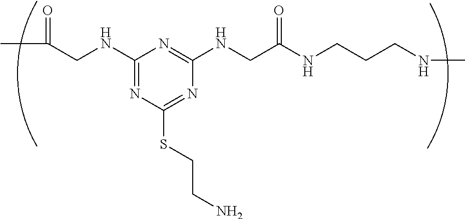 |
| Poly((4-((2- aminoethyl)thio)-6- ((1-((3-aminopropyl) amino)-4-methyl-1- oxopentan-2-yl) amino)-1,3,5-triazin- 2-yl)leucine) <10 KDa/>3 KDa Poly((4-((2- aminoethyl)thio)-6- ((1-((3-aminopropyl) amino)-4-methyl-1- oxopentan-2- yl)amino)-1,3,5-triazin- | 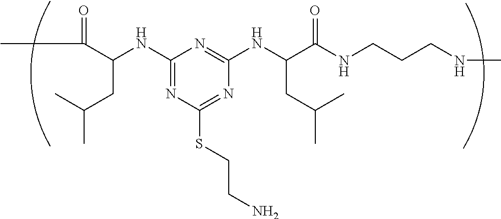 |
| 2-yl)leucine) | |
| <3 KDa/>1 KDa | |
| Poly((4-((6-amino-1-((3- aminopropyl)amino)-1- oxohexan-2-yl)amino)-6- (isopentylthio)-1,3,5- triazin-2-yl)lysine) <3 KDa/>1 KDa | 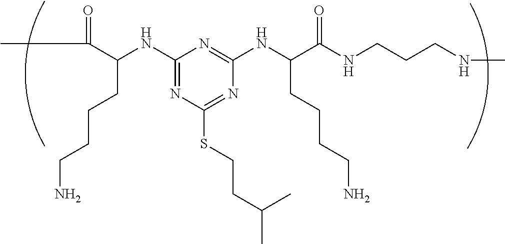 |

In an embodiment of the invention, the main chain polyamines are administered in an effective amount to achieve the desired therapeutic effect. The skilled artisan will be able to determine the effective amount of the main chain polyamines depending on the individual and the condition being treated.

In one embodiment of the invention, the main chain polyamines are used in the treatment all forms of mucositis, and are particularly effective when used to treat oral mucositis. Treatment includes prophylactic and therapeutic uses of the disclosed main chain polyamines and uses of the disclosed pharmaceutical compositions comprising main chain polyamines. Desired prophylactic effects include prevention and inhibition of mucositis, reduction in severity of mucositis, reduction in size of mucositis lesions and reduction in likelihood of developing mucositis through the application or administration of main chain polyamines or pharmaceutical compositions comprising main chain polyamines. Desired therapeutic effects include amelioration of the discomfort associated with the mucositis, and/or increased rate of healing of mucositis lesion.

In another embodiment, the main chain polyamines and pharmaceutical compositions comprising main chain polyamines can be used to treat all forms of SSIs. Treatment includes prophylactic and therapeutic uses of the disclosed main chain polyamines and uses of the disclosed pharmaceutical compositions comprising main chain polyamines. A desired prophylactic use is the immediate administration of main chain polyamines or pharmaceutical compositions comprising main chain polyamines to the surgical wound post-surgery in order to prevent and/or reduce the likelihood of developing a SSI. Another desired prophylactic use is the administration of main chain polyamines or pharmaceutical compositions comprising main chain polyamines prior to surgery in order to prevent and/or reduce the likelihood of developing a SSI. Desired therapeutic effects include the treatment of an existing SSI through the application or administration of main chain polyamines or pharmaceutical compositions comprising a main chain polyamine.

The main chain polyamines of the present invention may be administered alone or in a pharmaceutical composition comprising main chain polyamines. Suitable pharmaceutical compositions may comprise a main chain polyamine and one or more pharmaceutically acceptable excipients. The form in which the polymers are administered, for example, powder, tablet, capsule, solution, or emulsion, depends in part on the route by which it is administered. The main chain polyamines can be administered, for example, topically, orally, intranasally, by aerosol or rectally. Suitable excipients include, but are not limited to, are inorganic or organic materials such as gelatin, albumin, lactose, starch, stabilizers, melting agents, emulsifying agents, salts and buffers. Suitable pharmaceutically acceptable excipients for topical formulations such as ointments, creams and gels include, but are not limited to, commercially available inert gels or liquids supplemented with albumin, methyl cellulose, or a collagen matrix.

The main chain polyamines and pharmaceutical compositions comprising main chain polyamines can be administered alone or in combination with one or more additional drugs. Additional drugs administered in combination with the main chain polyamines and pharmaceutical compositions comprising main chain polyamines of the present invention include antibiotics and other compounds, including those used prophylactically and/or therapeutically for the treatment or prevention of mucositis and SSIs. The additional drugs may be administered concomitantly with the main chain polyamine or pharmaceutical compositions comprising main chain polyamines. The additional drugs may also be administered in series with the main chain polyamine or pharmaceutical compositions comprising main chain polyamines. The pharmaceutical composition comprising main chain polyamines may also further comprise a drug used prophylactically and/or therapeutically for the treatment or prevention of mucositis and SSIs.

### EXAMPLES

### Example 1

### In Vitro Studies

### Example 1-1

### Cytotoxicity Assay, RPTEC Cells

Mammalian cell cytotoxicity assays were performed using human renal proximal tubule epithelial cells (RPTEC—Cambrex CC-2553). Cells were plated at 3,000 cells/well (RPTEC) in 96-well plates and were incubated overnight at 37° C. The compounds were added to the wells, and the cells were incubated for 4 days. Alomar Blue was added to one set of plates and incubated for 4 hours. The plates were read when the compound was added (time zero) and at the end of the study. Fluorescence was read using 530 nm (excitation) and 590 nm (emission) according to the manufacturer's instructions. The 50% inhibitory concentration (IC₅₀) was calculated as 50% of the maximum signal minus the value at time zero.

Table 2 displays the renal proximal tubule epithelial cells IC₅₀ for selected compounds.

### Example 1-2

### Cytotoxicity Assay, Human Lung Epithelial Cells

Cytoxicity of the polymers towards human lung epithelial cells was performed using human lung epithelial Carcinoma cell line (A 549—ATCC # CCL-185). The cells were incubated for 96 hours at 7° C. with 5% CO₂ in a 96-well plate. CellTiter-Glo® (Promega) reagent was added to the plates. The plates were read by measuring the luminescence arising from luciferase catalyzed reaction of luciferin with ATP according to the manufacturer's suggested protocol. The concentration of ATP is directly proportional to cell viability; accordingly, higher luminescence measures high cell viability.

Table 2 displays the human lung epithelial cells IC₅₀ for selected compounds.

### Example 1-3

### Erythrocyte Lysis Assay

The compounds were incubated overnight at 37° C. in Dulbecco's phosphate-buffered saline containing fresh washed erythrocytes at a hematocrit of 1%. After incubation, the plates were centrifuged and the supernatant transferred to flat-bottomed 96-well plates. The supernatant was assayed using the QuantiChrom Hemoglobin kit according to the manufacturer's instructions. The IC₅₀ values were calculated using GraphPad Prism.

Table 2 displays the IC₅₀ values for selected compounds.

### Example 1-4

### Minimum Inhibitory Concentration Assay

The minimum inhibitory concentration (MIC) assay determines the lowest concentration of an antimicrobial agent required to inhibit the growth of test organisms after incubation. MIC assays were performed against an internal standard panel of organisms to identify compounds with antimicrobial activity. The MIC assay was subsequently repeated against other specialized microbial panels. Assays were conducted against the following clinically relevant microorganisms: Staphylococcus aureus subsp. aureus, Staphylococcus epidermis, Escherichia coli, Pseudomonas aeruginosa, and Haemophilius influenza. The compounds were tested for bacteriocidal activity, time course of killing, toxicity against tissue culture cells grown in vitro, and in some cases were tested for antimicrobial activity in vivo.

The MIC assays were performed according to the Performance Standards for Antimicrobial Susceptibility Testing, 2006, vol. M100-S15, Fifteenth Informational Supplement, NCCLS, 940 West Valley Road, Suite 1400, Wayne, Pa. 19087.

The polymers tested were dissolved in 0.85% saline to a final concentration of either 830 or 1000 μg/mL and the pH was adjusted to 7.0. The solution was then filter-sterilized through a 0.22 μm filter. Two-fold serial dilutions of polymer were prepared in Mueller-Hinton broth with cations aliquotted into 96-well microtiter plates. The plates were then inoculated with 5×10⁵ cells/mL of target organism and incubated 18-24 hours at 35° C. The optical density (OD) was read at 590 nm, and microorganism growth was scored (OD>0.1 is considered to be growth; OD<0.1 is considered to be growth inhibition). The MIC value is the lowest concentration of compound that inhibits growth; accordingly, a higher MIC value indicates less potency where a lower MIC valued indicated more potency.

MIC values of representative main chain polyamines against clinically relevant microorganisms are presented in Table 2.

TABLE 2
In vitro Results of Representative Main Chain Polyamines Cytotoxicity Assay [Kidney Epithelial
IC₅₀], Erythrocyte Lysis Assay [Hemolysis IC₅₀], and MIC values against Clinically Relevant Microorganisms

| | Cyto- | | Eryth- | | | | | |
|---|---|---|---|---|---|---|---|---|
| | toxicity | | rocyte | | Staphylo- | | Pseudo- | Haemoph- |
| | (Kidney | Lung | Lysis | Staphylococcus | coccus | | monas | ilus |
| | Epithelial | Epi | (Hemolysis | aureus subsp. | epidermis | Escherichia | aeruginosa | influenzae |
| Polymer Description | IC₅₀) | IC₅₀ | IC₅₀) | aureus (MIC) | (MIC) | coli (MIC) | (MIC) | (MIC) |
| 4,4′-Trimethylenedipiperidine/ | 1.5 | 3 | <6.25 | 8.0 | 2.0 | 8.0 | 16.0 | 32.0 |
| Glutaraldehyde >10K | | | | | | | | |
| 4,4′-dipiperidine/Glutaraldehyde | 1.5 | 5 | 11 | 1.0 | 0.3 | 4.0 | 8.0 | 64.0 |
| <10K | | | | | | | | |
| 4,4′-Trimethylenedipiperidine/1,4- | 10 | 57 | 4419 | 128.0 | 16.0 | 128.0 | 128.0 | 128.0 |
| cyclohexanedione (1:1) <10K | | | | | | | | |
| 4,4′-Trimethylenedipiperidine/2,6- | 1.5 | 23 | 3475 | 128.0 | 16.0 | 16.0 | 64.0 | 128.0 |
| Pyridine dicarboxaldehyde >10K | | | | | | | | |
| 4,4′-Trimethylenedipiperidine/ | 16 | 12 | 1310 | 128.0 | 128.0 | 128.0 | 128.0 | 128.0 |
| dibutylether heptamere | | | | | | | | |
| 4,4′-Trimethylenedipiperidine/ | 4 | 171 | 2485 | 128.0 | 128.0 | 128.0 | 128.0 | 128.0 |
| Glutaraldehyde (1:1.5) <10K | | | | | | | | |
| 4,4′-Trimethylenedipiperidine/ | 5 | 4 | 1923 | 8.0 | 2.0 | 4.0 | 16.0 | 128.0 |
| Glutaraldehyde (1:1.5) >10K | | | | | | | | |
| 4,4′-Trimethylenedipiperidine/ | 2 | 17 | 6400 | 128.0 | 16.0 | 32.0 | 128.0 | 32.0 |
| Glutaraldehyde (1:2.5) <10K | | | | | | | | |
| 4,4′-Trimethylenedipiperidine/ | 2 | 23 | 6400 | 128.0 | 16.0 | 32.0 | 128.0 | 64.0 |
| Glutaraldehyde (1:3.5) <10K | | | | | | | | |
| 4,4′-Trimethylenedipiperidine/ | 331 | 2859 | >6400 | 128.0 | 128.0 | 128.0 | 128.0 | 128.0 |
| Glutaraldehyde <10K | | | | | | | | |
| 4,4′-Trimethylenedipiperidine/ | 1.5 | 11 | 40 | 32.0 | 8.0 | 64.0 | 64.0 | 128.0 |
| Glyoxal >10K | | | | | | | | |
| 4,4′-Trimethylenedipiperidine/ | 1.5 | 11 | 33 | 16.0 | 8.0 | 16.0 | 32.0 | 128.0 |
| Isophthal dicarboxaldehyde >10K | | | | | | | | |
| 4,4′-Trimethylenedipiperidine/ | 1.5 | 17 | 36 | 64.0 | 8.0 | 64.0 | 128.0 | 128.0 |
| Terephthal dicarboxaldehyde >10K | | | | | | | | |
| 4,4′-Trimethylendipiperidine/dibutyl | 1.5 | 4 | 1476 | 64.0 | 4.0 | 8.0 | 128.0 | 16.0 |
| ether polymer (pyridine terminated)- | | | | | | | | |
| hydrogenation.HCl salt 1-3K | | | | | | | | |
| 4,4′-Trimethylendipiperidine/dibutyl | 1.5 | 4 | 3918 | 128.0 | 8.0 | 32.0 | 128.0 | 128.0 |
| ether polymer (pyridine terminated)- | | | | | | | | |
| hydrogenation.HCl salt 1-3K | | | | | | | | |
| 4,4′Trimethylenedipiperidine/dibutyl | 2 | 7 | 1596 | 128.0 | 8.0 | 16.0 | 128.0 | 32.0 |
| ether polymer (piperidine terminated)- | | | | | | | | |
| hydrogenation.HCl salt 1-3K | | | | | | | | |
| Glycidol modified poly4,4′- | 1 | 6 | 3200 | 8.0 | 2.0 | 16.0 | 16.0 | 128.0 |
| Trimethylenedipiperidine/ | | | | | | | | |
| Glutaraldehyde (1:1) | | | | | | | | |
| Poly(0.2:0.8:1 Me-PEG12- | 17 | — | >3200 | 16.0 | 4.0 | 64.0 | 128.0 | 128.0 |
| Piperidine:Dipiperidine:Glutaraldehyde); | | | | | | | | |
| <10K molecular weight cut | | | | | | | | |
| Poly(0.2:0.8:1 Me-PEG4- | 18 | — | >3200 | 32.0 | 2.0 | 16.0 | 32.0 | 128.0 |
| Piperidine:Dipiperidine:Glutaraldehyde); | | | | | | | | |
| <10K molecular weight cut | | | | | | | | |
| Poly(0.2:0.8:1 | 7 | — | >3200 | 2.0 | 0.5 | 16.0 | 64.0 | 128.0 |
| Piperidine:Dipiperidine:Glutaraldehyde); | | | | | | | | |
| <10K molecular weight cut | | | | | | | | |
| Poly(1:1 | 2 | 6 | 270 | 32.0 | 4.0 | 128.0 | 128.0 | 128.0 |
| Dipiperidine:Glutaraldehyde); | | | | | | | | |
| <10K molecular weight cut | | | | | | | | |
| Poly(4,4′-Trimethylenedipiperidine/ | 2 | 4 | 164 | 1.0 | 0.3 | 16.0 | 32.0 | 128.0 |
| Glutaraldehyde (1:1)), >10K | | | | | | | | |
| Poly(4,4′-Trimethylenedipiperidine/ | 2 | — | 203 | 2.0 | 0.5 | 32.0 | 32.0 | 128.0 |
| Glutaraldehyde (1:1)), <10K | | | | | | | | |
| Poly(4,4′-Trimethylenedipiperidine/ | 5 | 18 | 3200 | 1.0 | 0.3 | 16.0 | 32.0 | 128.0 |
| Glutaraldehyde/Piperidine | | | | | | | | |
| (0.8:1:0.2)), <10K | | | | | | | | |
| Poly(4,4′-Trimethylenedipiperidine/ | 1 | 5 | 108 | 0.5 | 0.1 | 4.0 | 8.0 | 128.0 |
| Glutaraldehyde/Piperidine | | | | | | | | |
| (0.8:1:0.2)), >10K | | | | | | | | |
| Poly(4,4′-Dipiperidine/ | 15.5 | 40.6 | >3200 | 16.0 | 2.0 | 128.0 | 128.0 | 128.0 |
| Glutaraldehyde/Piperidine-PEG12 | | | | | | | | |
| (0.8:1:0.2)) Mw <10K | | | | | | | | |
| Poly(4,4′-Dipiperidine/ | 6.8 | 18.8 | 344 | 8.0 | 2.0 | 64.0 | 128.0 | 128.0 |
| Glutaraldehyde/Piperidine-PEG12 | | | | | | | | |
| (0.8:1:0.2)) Mw >10K | | | | | | | | |
| Poly(4,4′-Dipiperidine/ | 23.8 | 81.4 | >3200 | 32.0 | 8.0 | 128.0 | 128.0 | 128.0 |
| Glutaraldehyde/Piperidine-PEG12 | | | | | | | | |
| (0.8:1:0.4)), Mw <10K | | | | | | | | |
| Poly(4,4′-Dipiperidine/ | 5.7 | 16.3 | 355 | 8.0 | 2.0 | 64.0 | 128.0 | 128.0 |
| Glutaraldehyde/Piperidine-PEG12 | | | | | | | | |
| (0.8:1:0.4)), Mw >10K | | | | | | | | |
| Poly(4,4′-Dipiperidine/ | 15.6 | 44.3 | >3200 | 16.0 | 4.0 | 128.0 | 128.0 | 128.0 |
| Glutaraldehyde/Piperidine-PEG4 | | | | | | | | |
| (0.8:1:0.2)), Mw <10K | | | | | | | | |
| Poly(4,4′-Dipiperidine/ | 5.8 | 17 | 305 | 8.0 | 2.0 | 64.0 | 128.0 | 128.0 |
| Glutaraldehyde/Piperidine-PEG4 | | | | | | | | |
| (0.8:1:0.2)), Mw >10K | | | | | | | | |
| Poly(4,4′-Dipiperidine/ | 7.1 | 13.5 | 1147 | 8.0 | 2.0 | 64.0 | 128.0 | 128.0 |
| Glutaraldehyde/Piperidine-PEG4 | | | | | | | | |
| (0.8:1:0.4)), Mw <10K | | | | | | | | |
| Poly(4,4′-Dipiperidine/ | 7.4 | 20 | 1383 | 16.0 | 2.0 | 64.0 | 128.0 | 128.0 |
| Glutaraldehyde/Piperidine-PEG4 | | | | | | | | |
| (0.8:1:0.4)), Mw >10K | | | | | | | | |
| Spermidin(N4-butanoic acid | 48 | 562 | 2678 | 128.0 | 32.0 | 128.0 | 128.0 | 128.0 |
| amide)/glyoxal polymer >10K | | | | | | | | |
| Spermidine(N4-butanoic acid | 2199 | 5119 | >6400 | 128.0 | 128.0 | 128.0 | 128.0 | 128.0 |
| amide)/glyoxal polymer <10K | | | | | | | | |
| Spermidine(N4-hexanoic acid | 688 | 4626 | >6400 | 128.0 | 128.0 | 128.0 | 128.0 | 128.0 |
| amide)/glyoxal polymer <10K | | | | | | | | |
| Spermidine(N4-propionic acid | 1542 | 5108 | >6400 | 128.0 | 128.0 | 128.0 | 128.0 | 128.0 |
| amide)/glyoxal polymer >10K | | | | | | | | |
| Poly (N,N′-dimethyl-1,6- | 2 | 23 | >3200 | 8.0 | 0.5 | 16.0 | 128.0 | >128 |
| diaminohexane/glutaraldehyde/piperidine) | | | | | | | | |
| [0.8:1:0.2] | | | | | | | | |
| Poly(N,N′-methyl-1,6- | >512 | 140 | >3200 | >128 | 64.0 | >128 | >128 | >128 |
| diaminohexane/glyoxal/piperidine) | | | | | | | | |
| [0.8:1:0.2] | | | | | | | | |
| Poly (N,N′-dimethyl-1,6- | 38 | 302 | >3200 | >128 | 32.0 | >128 | >128 | >128 |
| diaminohexane/succinicaldehyde/ | | | | | | | | |
| piperidine)[0.8:1:0.2] | | | | | | | | |
| Poly(Trimethylenedipiperidine/ | 1 | 0.5 | 71 | 4.0 | 1.0 | 4.0 | 16.0 | 32.0 |
| glutaraldehyde/piperidine{0.8:1:0.2}) | | | | | | | | |
| Glycidol(1:1) | 1 | 4 | 24 | 1.0 | 0.3 | 1.0 | 16.0 | 16.0 |
| modifiedPoly(Trimethylenedipiperidine/ | | | | | | | | |
| glutaraldehyde/piperidine{0.8:1:0.2}) | | | | | | | | |
| Poly(Trimethylenedipiperidine/glutaralde- | 3 | 1 | 455 | 16.0 | 2.0 | 8.0 | 64.0 | >128 |
| hyde/piperidine{0.8:1:0.2}) | | | | | | | | |
| Poly(Trimethylenedipiperidine/succinic | 29 | 8 | >3200 | >128 | 16.0 | >128 | >128 | >128 |
| aldehyde/piperidine{0.8:1:0.2}) | | | | | | | | |
| Poly(Bispiperidine/glutaraldehyde/PEG12 | 9 | 25 | >3200 | 8.0 | 0.5 | 32.0 | 128.0 | >128 |
| piperidine{0.6:1:0.4}) | | | | | | | | |
| Poly(Bispiperidine/glutaraldehyde/PEG4 | 3 | 10 | 144 | 2.0 | 0.5 | 16.0 | 64.0 | >128 |
| piperidine{0.6:1:0.4}) | | | | | | | | |
| Poly(Bispiperidine/glutaraldehyde/ | 4 | 8 | >3200 | 4.0 | 0.3 | 32.0 | 128.0 | >128 |
| piperidine) | | | | | | | | |
| Poly(N,N′-dibenzyl-1,6-diaminohexane/ | 11 | 29 | 116 | >128 | 32 | 64 | >128 | >128 |
| glutaraldehyde/piperidine)[0.8:1:0.2]; | | | | | | | | |
| +3K −10K fraction | | | | | | | | |
| Poly(1,6-diaminohexane/glutaraldehyde/ | 0.3 | 1 | >3200 | 0.5 | 0.1 | 2.0 | 4.0 | 64.0 |
| piperidine)[0.8:1:0.2]; +3K −10K fraction | | | | | | | | |
| Poly(Bispiperidine/glutaraldehyde/PEG12 | 3 | 2.6 | 77 | 1.0 | 0.3 | 8.0 | 32.0 | 128.0 |
| piperidine{0.8:1:0.2}); +10K fraction | | | | | | | | |
| Poly(Trimethylenedipiperidine/ | 1 | 0.5 | >3200 | 8.0 | 1.0 | 2.0 | 32.0 | 16.0 |
| glutaraldehyde/PEG4 piperidine{0.8:1:0.2}); | | | | | | | | |
| +3K −10K fraction | | | | | | | | |
| Poly(Trimethylenedipiperidine/ | 1 | 1 | 232 | 8.0 | 1.0 | 2.0 | 32.0 | 32.0 |
| glutaraldehyde/PEG12 piperi- | | | | | | | | |
| dine{0.8:1:0.2}); +3K −10K | | | | | | | | |
| fraction | | | | | | | | |
| Poly(Bispiperidine/glutaraldehyde/ | 4 | 5 | >3200 | 4.0 | 0.5 | 32.0 | 128.0 | >128 |
| piperidine{0.8:1:0.2}); | | | | | | | | |
| +3K −10K fraction | | | | | | | | |
| Poly(Trimethylenedipiperidine/ | 1 | 0.3 | 16 | 4.0 | 1.0 | 2.0 | 16.0 | 32.0 |
| glutaraldehyde/piperidine{0.8:1:0.2}); | | | | | | | | |
| +3K −10K fraction | | | | | | | | |
| Poly(N,N′-ethane-1,2-diyldipiperidine-4- | 5 | — | >3200 | | | | | |
| carboxamide/glutaraldehyde/piperidine) | | | | | | | | |
| Poly(N,N′-ethane-1,2-diyldipiperidine-4- | 8 | — | >3200 | | | | | |
| carboxamide/glutaraldehyde/PEG-4piperi- | | | | | | | | |
| dine) | | | | | | | | |
| Poly{6-((3-(4-(3-(1-(3- | 3 | 8 | 239 | 16 | 2 | 4 | 64 | 16 |
| aminopropyl)piperidin-4- | | | | | | | | |
| yl)propyl)piperidin-1- | | | | | | | | |
| yl)propyl)amino)-6-oxohexanoic acid} | | | | | | | | |
| <10 k/>3 k; 1.2:1 | | | | | | | | |
| Poly{6-((3-(4-(3-(1-(3- | 1 | 2 | 6 | 8 | 1 | 2 | 4 | >128 |
| aminopropyl)piperidin-4- | | | | | | | | |
| yl)propyl)piperidin-1- | | | | | | | | |
| yl)propyl)amino)-6-oxohexanoic acid} | | | | | | | | |
| >10 k; 1.2:1 | | | | | | | | |
| Poly{6-((3-(4-(3-aminopropyl) | >512 | >512 | 2659 | >128 | >128 | >128 | >128 | >128 |
| piperazin-1-yl)propyl)amino)-6- | | | | | | | | |
| oxohexanoic acid} | | | | | | | | |
| <10k/>3k; 1.24:1 | | | | | | | | |
| Poly{8-((3-(4-(3-(1-(3- | 4 | 15 | 42 | 128 | 4 | 4 | 128 | 16 |
| aminopropyl)piperidin-4- | | | | | | | | |
| yl)propyl)piperidin-1- | | | | | | | | |
| yl)propyl)amino)-8-oxooctanoic acid} | | | | | | | | |
| <10k/>3k; 1.2:1 | | | | | | | | |
| Poly{8-((3-(4-(3-(1-(3- | 0.4 | 2 | 4 | 16 | 2 | 4 | 16 | >128 |
| aminopropyl)piperidin-4- | | | | | | | | |
| yl)propyl)piperidin-1- | | | | | | | | |
| yl)propyl)amino)-8-oxooctanoic acid} | | | | | | | | |
| >10k; 1.2:1 | | | | | | | | |
| Poly{1-(3-(4-(3-(1-(3- | 7 | 46 | >3200 | 128 | 16 | 8 | >128 | 64 |
| aminopropyl)piperidin-4- | | | | | | | | |
| yl)propyl)piperidin-1-yl)propyl)-3-(4- | | | | | | | | |
| ureidobutyl)urea} <10k/>3k; 1.2:1 | | | | | | | | |
| Poly{1-(3-(4-(3-(1-(3- | 1 | 2 | 8 | 16 | 2 | 2 | 16 | >128 |
| aminopropyl)piperidin-4- | | | | | | | | |
| yl)propyl)piperidin-1-yl)propyl)-3-(4- | | | | | | | | |
| ureidobutyl)urea} >10k; 1.2:1 | | | | | | | | |
| Poly{5-((3-(4-(3-(1-(3- | 2 | 7 | 811 | 4 | 1 | 4 | 16 | 16 |
| aminopropyl)piperidin-4- | | | | | | | | |
| yl)propyl)piperidin-1- | | | | | | | | |
| yl)propyl)amino)-5-oxopentanoic | | | | | | | | |
| acid} <10k/>3k; 1.2:1 | | | | | | | | |
| Poly{5-((3-(4-(3-(1-(3- | 1 | 2 | 10 | 4 | 0.5 | 2 | 4 | >128 |
| aminopropyl)piperidin-4- | | | | | | | | |
| yl)propyl)piperidin-1- | | | | | | | | |
| yl)propyl)amino)-5-oxopentanoic | | | | | | | | |
| acid} >10k; 1.2:1 | | | | | | | | |
| Poly{1-(2-((6-aminohexyl)amino)-2- | 10 | NA | >3200 | >128 | 16 | >128 | >128 | >128 |
| oxoethyl)-4-(carboxymethyl)-1,4- | | | | | | | | |
| diazabicyclo[2.2.2]octane-1,4-diium | | | | | | | | |
| chloride} 1:1 | | | | | | | | |
| Poly{1-(2-((6-aminohexyl)amino)-2- | 13 | NA | 767 | 16 | 4 | 16 | 128 | >128 |
| oxoethyl)-4-(carboxymethyl)-1,4- | | | | | | | | |
| diazabicyclo[2.2.2]octane-1,4-diium | | | | | | | | |
| chloride} 1:1 | | | | | | | | |
| Poly{1-(2-((6-aminohexyl)amino)-2- | 24 | NA | >3200 | >128 | 32 | 32 | >128 | >128 |
| oxoethyl)-4-(carboxymethyl)-1,4- | | | | | | | | |
| diazabicyclo[2.2.2]octane-1,4-diium | | | | | | | | |
| chloride} 1:2 | | | | | | | | |
| Poly{1-(2-((8-aminooctyl)amino)-2- | 16 | NA | >3200 | >128 | 32 | >128 | >128 | >128 |
| oxoethyl)-4-(carboxymethyl)-1,4- | | | | | | | | |
| diazabicyclo[2.2.2]octane-1,4-diium | | | | | | | | |
| chloride} 1:1 | | | | | | | | |
| Poly{1-(2-((8-aminooctyl)amino)-2- | 24 | NA | >3200 | 128 | 16 | >128 | >128 | >128 |
| oxoethyl)-4-(carboxymethyl)-1,4- | | | | | | | | |
| diazabicyclo[2.2.2]octane-1,4-diium | | | | | | | | |
| chloride} 1:2 | | | | | | | | |
| Poly{(4-((2-aminoethyl)thio)-6-((2- | 9 | 69 | >3200 | >128 | >128 | >128 | >128 | >128 |
| ((3-aminopropyl)amino)-2- | | | | | | | | |
| oxoethyl)amino)-1,3,5-triazin-2- | | | | | | | | |
| yl)glycine} >10 KDa | | | | | | | | |
| Poly{(4-((2-aminoethyl)thio)-6-((1- | 284 | >512 | 1620 | >128 | >128 | >128 | >128 | >128 |
| ((3-aminopropyl)amino)-4-methyl-1- | | | | | | | | |
| oxopentan-2-yl)amino)-1,3,5-triazin- | | | | | | | | |
| 2-yl)leucine} <10 KDa/>3 KDa | | | | | | | | |
| Poly{(4-((2-aminoethyl)thio)-6-((1- | 214 | >512 | 2381 | >128 | >128 | >128 | >128 | >128 |
| ((3-aminopropyl)amino)-4-methyl-1- | | | | | | | | |
| oxopentan-2-yl)amino)-1,3,5-triazin- | | | | | | | | |
| 2-yl)leucine} <3 KDa/>1 KDa | | | | | | | | |
| Poly{(4-((2-aminoethyl)thio)-6-((2- | 57 | >512 | >3200 | >128 | >128 | >128 | >128 | >128 |
| ((3-aminopropyl)amino)-2- | | | | | | | | |
| oxoethyl)amino)-1,3 ,5-triazin-2- | | | | | | | | |
| yl)glycine} <3 KDa/>1 KDa | | | | | | | | |
| Poly{(4-((6-amino-1-((3- | 137 | >512 | >3200 | >128 | 32 | >128 | >128 | >128 |
| aminopropyl)amino)-1-oxohexan-2- | | | | | | | | |
| yl)amino)-6-(isopentylthio)-1,3,5- | | | | | | | | |
| triazin-2-yl)lysine} <3 KDa/>1 KDa | | | | | | | | |
| Poly(trimethylene dipiperidine-co- | >512 | NA | >3200 | >128 | >128 | >128 | >128 | >128 |
| piperidine-co-2,3-Butanedione) >10 KDa | | | | | | | | |
| Poly(trimethylenedi piperidine-co- | >512 | NA | >3200 | >128 | >128 | >128 | >128 | >128 |
| piperidine-co-2,3-Butanedione) <10 KDa | | | | | | | | |
| Poly(dipiperidine-co-piperidine-co- | >512 | NA | >3200 | >128 | >128 | >128 | >128 | >128 |
| 2,3-Butanedione) >10 KDa | | | | | | | | |
| Poly(dipiperidine-co-piperidine-co- | >512 | NA | >3200 | >128 | >128 | >128 | >128 | >128 |
| 2,3-Butanedione) <10 KDa | | | | | | | | |
| Poly(trimethylene | 95 | NA | >3200 | >128 | >128 | >128 | >128 | >128 |
| dipiperidine:piperidine-co-2,2- | | | | | | | | |
| Dimethyl-3,5-Hexanedione) | | | | | | | | |
| Poly(trimethylene | 32 | NA | 1030 | >128 | >128 | >128 | >128 | >128 |
| dipiperidine:piperidine-co-(±)- | | | | | | | | |
| Camphorquinone) | | | | | | | | |

### Example 2

### In Vivo Studies

### Example 2-1

### Toxicity—Maximum Tolerated Dose

Acute, 24 hour, toxicity studies to determine the maximum tolerated dose of a compound were carried out in male rats and mice of approximately 8-10 weeks of age. Animals were housed singly in standard polycarbonate cages and fed normal chow diets. Following one week of acclimation, compounds were administered in a single intraperitoneal (I.P.) or intravenous (I.V.) dose, typically in a PBS vehicle. The doses generally ranged from 1 mg/kg to as high as 400 mg/kg. Animals were observed for signs of pain, distress, and local or systemic signs of toxicity for one hour post-dosing, and then in 1 hour intervals for 6 hours after dosing. The following day at 24 hours post-dose, the animals were sacrificed and blood removed for serum chemistry analysis. Serum chemistry analyses performed include: ALT, AST, Creatinine and Urea Nitrogen. Major organs were also examined for abnormal signs.

Table 3 displays the Maximum Tolerated Dose (MTD) for select test compounds at select routes of administration.

TABLE 3
Maximum Tolerated Dose (MTD)

| | Animal | Route of | |
|---|---|---|---|
| Treatment | Model | Administration | MTD |
| Poly(0.2:0.8:1 Me-PEG12-Piperidine:Dipiperidine:Glutaraldehyde); | Rat | I.P. | ≧25 mg/kg |
| <10K molecular weight cut | | | |
| Poly(1:1 Dipiperidine:Glutaraldehyde); | Rat | I.P. | ≧25 mg/kg |
| <10K molecular weight cut | | | |
| Poly(Trimethylenedipiperidine/glutaraldehyde/PEG4 | Mice | I.P. | 25 mg/kg |
| piperidine{0.8:1:0.2}); | | | |
| +3K −10K fraction | | | |
| Poly(Trimethylenedipiperidine/glutaraldehyde/PEG12 | Mice | I.P. | 1 mg/kg |
| piperidine{0.8:1:0.2}); | | | |
| +3K −10K fraction | | | |
| Poly(Bispiperidine/glutaraldehyde/piperidine | Mice | I.V. | 10 mg/kg |
| {0.8:1:0.2}); +3K −10K fraction | | | |

### Example 3

### Synthesis of Main Chain Polyamines

### Example 3-1

### Synthesis of Poly(1,3-bis(1,6-dimethylenepyridine)piperidine-4-yl)propane)

One gram (1 g) of 4,4′-trimethylene dipiperidine was dissolved in 30 mL of tetrahydrofuran (THF) . To this solution 642 mg of 2,6 pyridine dicarbaldehyde was added. The resulting reaction mixture was stirred at ambient temperature for 2 hours. 4.03 g of sodium triacetoxyborohydride was added to the reaction mixture and stirred at ambient temperature for 18 hours; the solvent was removed under reduced pressure. The residue was dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-2

### Synthesis of Poly(1,3-bis(1,5-dimethybenzene)piperidin-4-yl)propane)

One gram (1 g) of 4,4′-trimethylene dipiperidine was dissolved in 30 mL of THF. To this solution 638 mg of isophtalaldehyde was added. The resulting reaction mixture was stirred at ambient temperature for 2 hours. 4.03 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-3

### Synthesis of Poly(1,3-bis N-(1,4-dimethybenzene)piperidin-4-yl)propane)

One gram (1 g) of 4,4′-trimethylene dipiperidine was dissolved in 30 mL of THF. To this solution 638 mg of terephthaldicarbaldehyde was added. The resulting reaction mixture was stirred at ambient temperature for 2 hours. 4.03 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-4

### Synthesis of Poly(1,3-bis N-(dimethylene)piperidin-4-yl)propane)

One gram (1 g) of 4,4′-trimethylene dipiperidine was dissolved in 30 mL of THF. To this solution 441 mg of a 40 wt. % aqueous Glyoxal was added. The resulting reaction mixture was stirred at ambient temperature for 2 hours. 4.03 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-5

### Synthesis of Poly(1,3-bis(1,4-cyclohexyl)piperidine-4-yl)propane)

One gram (1 g) of 4,4′-trimethylenedipiperidine was dissolved in 30 mL of THF. To this solution 553 mg 1,4-cyclohexanedione was added. The resulting reaction mixture was stirred at ambient temperature for 2 hours. 4.03 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-6

### Synthesis of Poly(l,3-bis(1-pentylpiperidin-4-yl)propane

One gram (1 g) of 4,4′-trimethylenedipiperidine was dissolved in 30 mL of THF. To this solution 537 g of a 70 wt. % aqueous glutaraldehyde solution was added. The resulting reaction mixture was stirred at ambient temperature for 2 hours. 8.05 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-7

### Synthesis Piperidine end Capped poly(1,3-bis(1-pentylpiperidin-4-yl)propane

One gram (1 g) of 4,4′-trimethylenedipiperidine was dissolved in 30 mL of THF. To this solution 506 mg of piperidine was added, followed by 1.9 g of a 50 wt. % solution of glutaraldehyde. The resulting reaction mixture was stirred at ambient temperature for 2 hours. 8.05 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-8

### Synthesis of Poly((1,1′-dipentyl)-4,4-dipiperidine)

One gram (1 g) of 4,4′-bipiperidine was dissolved in 30 mL of THF. To this solution 774 mg of glutaraldehyde as 70% aqueous solution was added. The resulting reaction mixture was stirred at ambient temperature for 2 hours. 5.04 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-9

### Synthesis of piperidine end capped poly(1,1′-dipentyl)-4,4-dipiperidine)

One gram (1 g) of 4,4′-bipiperidine was dissolved in 30 mL of THF. To this solution 126 mg of piperidine was added followed by 1.49 g of a 50 wt. % aqueous glutaraldehyde solution. The resulting reaction mixture was stirred at ambient temperature for 2 hours. 6.30 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-10

### Synthesis of 4-(tetraethyleneglycol amidomethyl)-piperidine end capped poly(l,3-bis(1-pentylpiperidin-4-yl)propane

### Example 3-10(a): Synthesis of 4-(tetraethyleneglycol amidomethyl)-piperidine ((N-piperidin-4-ylmethyl)-2,5,8,11-tetraoxatetradecan-14-amide)

To 1.05 g of commercial 4,7,10,13-tetraoxatetradecanoic acid, 2,5-dioxo-1-pyrrolindinyl ester in 10 mL dimethylformamide (DMF), 1.32 mL triethylamine followed by 675 mg of 1-boc-4-(aminomethyl) piperidine was added. The reaction mixture was stirred overnight and the DMF was evaporated. The crude reside was purified through silica with a gradient from neat dichloromethane to 10% (v/v) methanol/dichloroethane.

860 mg of tert-butyl 4-(14-oxo-2,5,8,11-tetraoxa-15-azahexdecan-16-yl)piperidine-1-carboxylate was dissolved in 10 mL dioxane and 4 mL of 4M hydrochloric acid in dioxane was added. The reaction mixture was stirred for 2 hours; the dioxane was evaporated. The resulting residue was mixed with 50 mL water. The aqueous solution was mixed with 4M sodium hydroxide and the resulting precipitate was extracted into 50 mL dichloromethane. The water phase was extracted with 50 mL dichloromethane twice more. The combined organic phase was dried over magnesium sulfate and the solvent evaporated.

### Example 3-10(b)

### Synthesis of 4-(tetraethyleneglycol amidomethyl)-piperidine end capped poly(1,3-bis(1-pentylpiperidin-4-yl)propane

1.34 g of 4,4′-trimethylenedipiperdine was dissolved in 30 mL of THF. To this solution, 530 mg of 4-(tetraethyleneglycol amidomethyl)-piperidine (Example 3-10(a)), was added, followed by 1.59 g of a 50 wt. % aqueous solution of glutaraldehyde. The resulting reaction mixture was stirred at ambient temperature for 2 hours. 6.74 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-11

### Synthesis of 4-(dodecaethyleneglycol amidomethyl)-piperidine end capped poly(l,3-bis(1-pentylpiperidin-4-yl)propane)

### Example 3-11(a)

### Synthesis of 4-(dodecaethyleneglycol amidomethyl)-piperidine (N-(piperidin-4-ylmethyl)-2,5,8,11,14,17,20.23,26,29,32,35-dodecaoxaoctatriacontain-38-amide)

To 910 g of commercial 1-[(38-oxo-2,5,8,11,14,17,20,23,26,29,32,35-dodecaoxaoctatriacontan-38-yl)oxy]pyrrolidine-2,5-dione in 10 mL DMF 555 μL triethylamine was added, followed by 284 mg of 1-boc-4-(aminomethyl) piperidine. The reaction was stirred over night; the DMF evaporated. The crude residue was purified through silica with a gradient from neat dichloromethane to 10% (v/v) methanol/dichloromethane.

990 mg of tert-butyl 4-(38-oxo-2,5,8,11,14,17,20,23,26,29,32,35-dodecaoxa-39-azatetracontan-40-yl)piperidine-1-carboxylate was dissolved in 10 mL dioxane and 4 mL of 4M hydrochloric acid in dioxane was added. The reaction mixture was stirred for 2 hours, after which the dioxane was evaporated. The resulting residue was mixed with 50 mL water. The aqueous solution was mixed with 4M sodium hydroxide and the resulting precipitate was extracted into 50 mL dichloromethane. The water phase was extracted with 50 mL dichloromethane twice more. The combined organic phase was dried over magnesium sulfate and the solvent evaporated.

### Example 3-11(b)

### Synthesis of 4-(dodecaethyleneglycol amidomethyl)-piperidine end capped poly(1,3-bis(1-pentylpiperidin-4-yl)propane

307 mg of 4,4′-trimethylenedipiperdine was dissolved in 30 mL of THF. To this solution, 250 mg of 4-(dodecaethyleneglycol amidomethyl)-piperidine (Example 3-11(a))was added followed by 366 mg of a 50 wt. % aqueous solution of glutaraldehyde as. The resulting reaction mixture was stirred at ambient temperature for 2 hours. 1.5 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-12

### Synthesis of 4-(triethyleneglycol amidomethyl)-piperidine end capped poly((l,1′-dipentyl)-4,4′-dipiperidine)

100 mg of 4,4′-bispiperdine was dissolved in 15 mL of THF. To this solution was added 50 mg of 4-(triethyleneglycol amidomethyl)-piperidine, followed by 106 mg of a 70 wt. % aqueous solution of glutaraldehyde. The resulting reaction mixture was stirred at ambient temperature for 2 hours. 630 g of sodium triacetoxyborohydride was added to the reaction mixture and stirred at ambient temperature for 18 hours; the solvent was removed under reduced pressure. The solids were dissolved in 10 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-13

### Synthesis of 4-(dodecaethyleneglycol amindomethyl)-piperidine end capped poly((1,l -dipentyl)-4,4-dipiperidine)

100 mg of 4,4′-bispiperdine was dissolved in 15 mL of THF. To this solution, 102 mg of 4-(4-(dodecaethylenegly amidomethyl)-piperidine (Example 3-11(a)) was added, followed by 106 mg of a 70 wt. % aqueous solution of glutaraldehyde as 70%. The resulting reaction mixture was stirred at ambient temperature for 2 hours. 630 mg of sodium triacetoxyborohydride was added to the reaction mixture and stirred at ambient temperature for 18 hours; the solvent was removed under reduced pressure. The solids were dissolved in 10 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-14

### Synthesis of Poly(1,3-bis(1-butylpiperidin-4-yl)propane

One gram (1 g) of succinaldehyde bis(dimethyl acetal) was stirred at room temperature with 1.06 mL of water and 3 mL of acetic acid for 90 minutes. One gram (1 g) of 4,4′-trimethylenedipiperdine and 101 mg of piperidine were dissolved in 30 mL THF. The succinaldehyde solution was added to the reaction. The resulting reaction mixture was stirred at ambient temperature for 2 hours. 5.04 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-15

### Synthesis of piperidine end capped poly(N¹,N⁶-dimethyl-N¹,N⁶-dipentylhexane-1,6-diamine)

One gram (1 g) of N,N′dimethyl-1,6-hexanedimine was dissolved in 35 mL of THF. To this solution, 1.24 g of glutaraldehyde as 70% aqueous solution followed by 151 mg of piperidine was added. The resulting reaction mixture was stirred at ambient temperature for 2 hours. 5.87 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-16

### Synthesis of piperidine end capped poly(N¹,N⁶-dimethyl-N¹,N⁶-dibutylhexane-1,6-diamine)

1.54 g of succinaldehyde bis(dimethyl acetal) (1g) was stirred at room temperature with 1.54 mL of water and 3 mL of acetic acid for 120 minutes. One gram (1 g) of 4,4′-N,N′dimethyl-1,6-hexane diamine and 148 mg of piperidine were dissolved in 30 mL THF. The succinaldehyde solution was added to the reaction. The resulting reaction mixture was stirred at ambient temperature for 2 hours. 7.33 g of sodium triacetoxyborohydride was added to the reaction mixture and stirred at ambient temperature for 18 hours; the solvent was removed under reduced pressure. The solids were dissolved in 100 mL of water and the pH was adjusted to 14 with 4M sodium hydroxide at which time a white precipitate formed. The reaction mixture was filtered and the solids were dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-17

### Synthesis of piperidine end capped poly(N1,N6-dimethyl-N1,N6-diethylhexane-1,6-diamine)

One gram (1 g) of N,N′dimethyl-1,6-hexanedimine was dissolved in 30 mL of THF. To this solution, 1.25 g of glyoxal as 40% aqueous solution followed by 148 mg of piperidine was added. The resulting reaction mixture was stirred at ambient temperature for 2 hours. 7.33 g of sodium triacetoxyborohydride was added to the reaction mixture and stirred at ambient temperature for 18 hours; the solvent was removed under reduced pressure. The solids were dissolved 1.2M hydrochloric acid and the pH was then adjusted to 14 with 4 M sodium hydroxide at which time an oily substance was separated. The oily substance was extracted into methylene chloride and the solvent was evaporated. The residue was dissolved in 1.2M hydrochloric acid to a pH of 1. The resulting solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-18

### Synthesis of poly(N-(4-ethylaminobutyl)-N-(3-ethylaminopropyl)propanamide)

### Example 3-18(a)

### Synthesis of N1,N8-Di(tert-butoxycarbonyl)spermidine

24.56 g of 1,1′-carbonydiimadazole was dissolved in 500 mL of toluene. 1.7 g of potassium hydroxide and 11.25 g t-butanol were added to the solution and the reaction mixture was heated to 60° C. for 3 hours. 11 g of spermidine was added and the reaction mixture continued heating at 60° C. for an additional 3 hours. The reaction mixture then cooled to ambient temperature. All solvent was removed by roto-vap under vacuum and the solids were dissolved in 200 mL of water. The aqueous solution was extracted four times with 100 mL of methylene chloride. The extracts were combined and dried over magnesium sulfate for 1 hour. The solution was filtered and all solvent removed by roto-vap under vacuum. The yield was 20.22 g of N1,N8-Di(tert-butoxycarbonyl)spermidine.

### Example 3-18(b)

### Synthesis of N-(4-N-(tert-butoxycarbony)aminobutyl)-N-(3-N-(tert-butoxycarbonyl)aminopropyl)-propanamide

856 mg of propionic acid was dissolved in 40 mL DMF. To the solution 1.72 g of 1-hydroxybenzotriazole was added. The reaction flask was pumped down with a vacuum and a nitrogen atmosphere was introduced; vacuuming and introduction of the nitrogen atmosphere was repeated. The reaction was cooled to 0° C. with an ice water bath. 1.61 g diisopropylcarbodiimide was added and the reaction mixture was stirred for 1 hour at 0° C. 4 g of N1,N8-Di(tert-butoxycarbonyOspermidine (Example 3-18(a)) was added, followed immediately by the addition of 1.94 g of diisopropylethylamine . The reaction mixture was allowed to warm to ambient temperature over 18 hours. All solvent was removed by roto-vap under vacuum. 100 mL of diethyl ether was added, forming a white precipitate. The solution was filtered and the organics were extracted with 25 mL brine (3 times) followed by 25 mL of 10% citric acid and finally by 25 mL 4M sodium hydroxide before being dried over magnesium sulfate for 1 hour. The solution was filtered and all solvent removed by roto-vap under vacuum. Further purification was performed by flash chromatography using a 50 g silica column (1CV=66 mL, 1 fraction=22 mL, 50 mL/min).

The column was primed with 3CV 5% ethyl acetate/95% hexane. The gradient was held at 5% ethyl acetate/95% hexane for 1CV before increasing to 100% ethyl acetate over 10CV and held at 100% ethyl acetate for 2CV. The product was found by TLC in fractions 23-31. The fractions were combined and all solvent was removed by roto-vap under vacuum. The yield was 1.98 g N-(4-N-(tert-butoxycarbony)aminobutyl)-N-(3-N-(tert-butoxycarbonyl)aminopropyl)-propanamide.

### Example 3-18(c)

### Synthesis of N-(4-aminobutyl)-N-(3-ethylaminopropyl)-propanamide

1.98 g of N-(4-N-(tert-butoxycarbonyl)aminobutyl)-N-(3-N-(tert-butoxycarbonyl)aminopropyl)propanamide was dissolved in 20 mL 4 M hydrochloric acid in 1, 4-dioxane and stirred for 2 hours at ambient temperature. All solvent was removed by roto-vap under vacuum and dissolved in 75 mL of water. The aqueous solution was adjusted to pH 14 with 4M sodium hydroxide and extracted three times with 50 mL methylene chloride. The organics are combined and dried over magnesium sulfate for 1 hour. The solution was filtered and all solvent was removed by roto-vap under vacuum. The yield was 456 mg of N-(4-aminobutyl)-N-(3-ethylaminopropyl)-propanamide.

### Example 3-18(d):

### Poly(N-(4-ethylaminobutyl)-N-(3-ethylaminopropyl)-propanamide)

456 mg of N-(4-aminobutyl)-N-(3-ethylaminopropyl)-propanamide was dissolved in 3 mL of methanol. 211 mg of glyoxal as 40% aqueous solution was added to the solution and stirred for 18 hours at ambient temperature. 5 mL of methanol was added to the reaction mixture and was heated to 45° C. 644 mg of sodium borohydride was slowly added. Violent bubbling occurred. Heating was continued for 1.5 hours before allowing it to cool to ambient temperature. All solvent was removed by roto-vap under vacuum. 25 mL of water was added to the reaction and concentrated hydrochloric acid was added until soluble. 4M sodium hydroxide was added to obtain a pH of 14. The aqueous phase was extracted several times with dichloromethane. The combined extracts were dried over MgSO₄ and the solvent was evaporated. The crude material was treated with 1M hydrochloric acid, after which an emulsion formed. The emulsion was dissolved by diluting the mixture with water. The pH was kept below 1. The reaction solution was passed through a 10 KDa Macrosep filtration device by centrifugation at 5,000 rpm for 30 minutes. The retained material was diluted with water and the centrifugation was repeated four times. This method yields fractions of >10 KDa and <10 KDa. Each fraction was frozen and placed on the lyophilizer to dry. A fluffy white solid was obtained for each fraction.

### Example 3-19

### Synthesis of poly(N-(4-ethylaminobutyl)-N-(3-ethylaminopropyl) butanamide)

### Example 3-19(a)

### Synthesis of N-(4-N-(tert-butoxycarbonyl)aminobutvl)-N-(3-N-(tert-butoxycarbonvI)aminopropyl)-butanamide

1.02 g of butyric acid was dissolved in 40 mL DMF; 1.72 g of 1-Hydroxybenzotriazole was added. The reaction flask was pumped down with vacuum and a nitrogen atmosphere was introduced (repeated twice). The reaction was cooled to 0° C. with an ice water bath. 1.61 g of diisopropylcarbodiimide was added and the reaction mixture was stirred for 1 hour at 0° C. 4 g of N1,N8-di(tert-butoxycarbonyl)spermidine was added followed immediately by the addition of 1.94 g of diisopropylethylamine. The reaction mixture was allowed to warm to ambient temperature over 18 hours. All solvent was removed by roto-vap under vacuum. 100 mL of diethyl ether was added, forming a white precipitate. The solution was filtered and the organics were extracted with 25 mL brine (repeated three times, followed by 25 mL of 10% citric acid and then 25 mL of 4M sodium hydroxide. The reaction mixture was then dried over magnesium sulfate for 1 hour. The solution was filtered and all solvent was removed by roto-vap under vacuum. Further purification was performed by flash chromatography using a 50 g silica column (1CV =66 mL, 1 fraction =22 mL, 50 mL/min). The column was primed with 3CV 5% ethyl acetate/95% hexane. The gradient was held at 5% ethyl acetate/95% hexane for 1CV before increasing to 100% ethyl acetate over 10CV and held at 100% ethyl acetate for 2CV. The product was found by TLC in fractions 23-31. The fractions were combined and all solvent was removed by roto-vap under vacuum. The yield was 2.01 g of N-(4-N-(tert-butoxycarbony)aminobutyl)-N-(3-N-(tert-butoxycarbonyl)aminopropyl)-butanamide.

### Example 3-19(b)

### Synthesis of N-(4-aminobutyl)-N-(3-aminopropyl)butyramide

2.01 g of N-(4-N-(tert-butoxycarbony)aminobutyl)-N-(3-N-(tert-butoxycarbonyl)aminopropyl)-butanamide was dissolved in 220 mL of 4M hydrochloric acid in 1, 4-dioxane and stirred for 2 hours at ambient temperature. All solvent was removed by roto-vap under vacuum. The reaction mixture was dissolved in 75 mL of water. The aqueous solution was adjusted to pH 14 with 4M sodium hydroxide and extracted with 50 mL methylene chloride (repeated three times). The organics were combined and dried over magnesium sulfate for 1 hour. The solution was filtered and all solvent was removed by roto-vap under vacuum. The yield was 529 mg of N-(4-aminobutyl)-N-(3-aminopropyl)butyramide

### Example 3-19(c)

### Synthesis of poly(N-(4-ethylaminobutyl)-N-(3-ethylaminopropyl) butanamide)

529 mg of N-(4-aminobutyl)-N-(3-aminopropyl)butyramide was dissolved in 4 mL of methanol. 229 mg of glyoxal 40% aqueous solution was added and stirred for 18 hours at ambient temperature. 5 mL of methanol was added to the reaction and it was heated to 45° C. 698 mg of sodium borohydride was slowly added. Violent bubbling occurred. Heating was continued for 1.5 hours before allowing the reaction to cool to ambient temperature. All solvent was removed by roto-vap under vacuum. 25 mL of water was added to the reaction and hydrochloric acid was added until soluble. 4 M sodium hydroxide was added to obtain a pH of 14 when a white precipitate was observed. The reaction was filtered and dissolved in 1.2 M hydrochloric acid until the pH is 1. This solution was passed thru a 10 KDa Macrosep filtration device by centrifugation at 5,000 rpm for 30 minutes. The retained material was diluted with water and the centrifugation was repeated four times. This method yields fractions of >10 KDa and <10 KDa. Each fraction was frozen and placed on the lyophilizer to dry. A fluffy white solid was obtained for each sample.

### Example 3-20

### Synthesis of poly(N-(4-ethylaminobutyl)-N-(3-ethylaminopropyl) hexanamide)

### Example 3-20(a)

### Synthesis of tert-butyl (3-(N-(4-((tert-butoxycarbonyl)amino) butyl)hexanamido)propyl)carbamate

336 mg of hexanoic acid was dissolved in 10 mL DMF; 430 mg of 1-hydroxybenzotriazole was added to the solution. The reaction flask was pumped down with vacuum and a nitrogen atmosphere was introduced (repeated twice) . The reaction was cooled to 0° C. with an ice water bath. 401 mg diisopropylcarbodiimide was added and the reaction mixture was stirred for 1 hour at 0° C. One gram (1 g) of N1,N8-di(tert-butoxycarbonyl)spermidine was added followed immediately by 486 mg of diisopropylethylamine. The reaction mixture was allowed to warm to ambient temperature over 18 hours. All solvent was removed by roto-vap under vacuum and 100 mL diethylether was added, forming a white precipitate. The solution was filtered and the organics were extracted with 25 mL brine (repeated three times) followed by 25 mL of 10% citric acid and then 25 mL 4M sodium hydroxide. The reaction mixture was dried over magnesium sulfate for 1 hour. The solution was filtered and all solvent was removed by roto-vap under vacuum. Further purification was performed by flash chromatography using a 50 g silica column (1CV =66 mL, 1 fraction=22 mL, 50 mL/min). The gradient was held at 5% ethyl acetate/95% hexane for 1CV before increasing to 100% ethyl acetate over 10CV and held at 100% ethyl acetate for 2CV. The resulting product was found by TLC in fractions 24-35. These fractions were combined and all solvent was removed by roto-vap under vacuum. The yield was 880 mg of tert-butyl (3-(N-(4-((tert-butoxycarbonyl)amino)butyl)hexanamido)propyl)carbamate.

### Example 3-20(b)

### Synthesis of N-(4-aminobutyl)-N-(3-aminopropyl)hexanamide

800 mg of tert-butyl (3-(N-(4-((tert-butoxycarbonyl)amino)butyl)hexanamido) propyl)carbamate was dissolved in 10 mL of 4M hydrochloric acid in 1,4-dioxane and stirred for 2 hours at ambient temperature. All solvent was removed by roto-vap under vacuum. The resulting reaction mixture was dissolved in 75 mL of water. The aqueous solution was adjusted to pH 14 with 4M sodium hydroxide and extracted with 50 mL methylene chloride (repeated three times). The organics were combined and dried over magnesium sulfate for 1 hour. The solution was filtered and all solvent was removed by roto-vap under vacuum. The yield was 280 mg of N-(4-aminobutyl)-N-(3-aminopropyl)hexanamide.

### Example 3-20(c)

### Synthesis of poly(N-(4-ethylaminobutyl)-N-(3-ethylaminopropoy)-hexanamide

280 mg of N-(4-aminobutyl)-N-(3-aminopropyl)hexanamide was dissolved in 2 mL of methanol. 107 mg of glyoxal 40% aqueous solution in water was added and stirred for 18 hours at ambient temperature. 5 mL of methanol was added to the reaction and it was heated to 45° C. 327 mg of sodium borohydride was slowly added. Violent bubbling occurred. Heating was continued for 1.5 hours before allowing the reaction to cool to ambient temperature. All solvent was removed by roto-vap under vacuum. 25 mL of water was added to the reaction and hydrochloric acid was added until it was soluble. 4M sodium hydroxide was added to obtain a pH of 14, a white precipitate was observed. The reaction was filtered and dissolved in 1.2M hydrochloric acid until the pH was 1. This solution was passed through a 10 KDa Macrosep filtration device by centrifugation at 5,000 rpm for 30 minutes. The retained material was diluted with water and the centrifugation was repeated four times. This method yields fractions of >10 KDa and <10 KDa. Each fraction was frozen and placed on the lyophilizer to dry. A fluffy white solid was obtained for each sample.

### Example 3-21

### Synthesis of piperidine end capped poly(N′,N⁶-dibenzyl-N′,N⁶-dipentylhexane-1,6-diamine)

### Example 3-21(a)

### Synthesis of N1, N6-dibenzylhexane-1,6-diamine.

4.98 g of benzaldehyde and 2.65 g of 1,6-diaminohexane were dissolved 100 mL of methanol, 10 g of magnesium sulfate was added. The slurry was stirred overnight and then filtered. The filtrate was treated with 3.45 g of NaBH₄ and the reaction was left over night. The methanol was evaporated and the residue was dissolved in a mixture of water and dichloromethane. The water phase was re-extracted twice with dichloromethane and the magnesium sulfate dried combined organic phase evaporated. The crude product was purified through silica with a gradient from neat ethyl acetate to 10% methanol/triethylamine(1:1) in ethyl acetate. The yield was 4.2 g of N1, N6-dibenzylhexane-1,6-diamine.

### Example 3-21(b)

### Synthesis of piperidine end capped poly(N′,N⁶-dibenzyl-N¹,N⁶-dipentylhexane-1,6-diamine

One gram (1 g) of N1, N6-dibenzylhexane-1,6-diamine was dissolved in 17 mL of THF. 703 mg of a 50 wt. % aqueous glutaraldehyde solution and 72 mg of piperidine was added. The reaction mixture was stirred at ambient temperature for 2 hours. 2.9 g of sodium triacetoxyborohydride was added. The resulting reaction mixture was stirred at ambient temperature for 18 hours. The solvent was evaporated and the residue was dissolved in 100 mL of water and the pH was adjusted to 14 with 4M sodium hydroxide, a white precipitate formed. The reaction mixture was filtered and the residue was dissolved in 1.2M hydrochloric acid to a pH of 1. The solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-22

### Synthesis of piperidine end capped poly(N¹,N⁶-dipentylhexane-1,6-diamine)

113 mg of the >10K fraction of poly(N¹,N⁶-dibenzyl-N¹,N⁶-dipentylhexane-1,6-diamine) was dissolved in 7 mL of methanol/1 ml aqueous hydrochloric acid. 210 mg of 10% Pd/C was added. A Parr pressure vessel was assembled and the vessel was evacuated. The vacuum was released with hydrogen and the pump-release was repeated twice before pressurizing the Parr pressure vessel to 10 bars. The reaction was heated to 60° C. and left for 48 hours. After releasing the pressure in the Parr pressure vessel, the catalyst, Pd/C, was filtered off through Celite. The methanol was evaporated. The residue was taken up in 1.2M hydrochloric acid, frozen and placed on a lyophilizer to freeze dry.

### Example 3-23

### Synthesis of pyridine end capped poly(4,4′-propane-1,3-diylbis[1-(4-butoxybutyl)piperidine)

517 mg of the <3 KDa/>1 KDa fraction of poly(1-(4-butoxybutyl)-4-(3-{1-[4-(pentyloxy)butyl]pyridinium-4-yl]propyl)pyridinium dichloride) was dissolved in 40 mL of methanol. 177 mg of platinum oxide was added. A Parr pressure vessel was assembled and hydrogen was bubbled through the reaction mixture for 5 minutes before pressurizing the Parr pressure vessel to 40 bars. The reaction was heated to 60° C. and left for 48 hours. After releasing the pressure in the Parr pressure vessel, the catalyst, platinum black, was filtered off by passing the reaction mixture through Celite. The methanol was evaporated. The solids were dissolved in aqueous hydrochloric acid and the pH was adjusted to 14 with 8M sodium hydroxide; a white precipitate formed. The reaction was filtered and the solids were re-dissolved in methanol. The solution was filtered again and evaporated. The residue was taken up in 1.2M hydrochloric acid, frozen and placed on a lyophilizer to freeze dry.

### Example 3-24

### Synthesis of piperidine end capped poly(4,4′-propane-1,3-diylbis[1-(4-butoxybutyl)piperidine])

517 mg of the <3 KDa/>1 KDa fraction of poly(l-(4-butoxybutyl)-4-(3-{1-[4-(pentyloxy)butyl]pyridinium-4-yl}propyl)pyridinium dichloride) was dissolved in 40 mL of methanol and 2 mL 1.2M aqueous hydrochloric acid. 177 mg of platinum oxide was added. A Parr pressure vessel was assembled and hydrogen was bubbled through the reaction mixture for 5 minutes before pressurizing the Parr pressure vessel to 40 bars. The reaction was heated to 60° C. and left for 48 hours. After releasing the pressure in the Parr pressure vessel, the catalyst, platinum black, was filtered off by passing the reaction mixture through Celite. The methanol was evaporated. The solids were dissolved in aqueous hydrochloric acid and the pH was adjusted to 14 with 8M sodium hydroxide; a white precipitate formed. The reaction was filtered and the solids were redissolved in methanol. The solution was filtered again and evaporated. The residue was taken up in 1.2M hydrochloric acid, frozen and placed on a lyophilizer to freeze dry.

### Example 3-25

### Synthesis of Poly(trimethylene dipiperidine-co-piperidine-co-2,3-Butanedione)

One gram (1 g) of 4,4′-trimethylenedipiperdine was dissolved in 30 mL of THF. 511 mg of 2,3-butanedione and 101 mg of piperidine was added. The reaction mixture was stirred at ambient temperature for 2 hours. 5.0 g of sodium triacetoxyborohydride was added. The resulting reaction mixture was stirred at ambient temperature for 18 hours. . The solvent was evaporated and the residue was dissolved in 100 mL of water. The pH was adjusted to 14 with 4M sodium hydroxide, a white precipitate formed. The reaction mixture was filtered and the residue was dissolved in 1.2M hydrochloric acid to a pH of 1. The solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-26

### Synthesis of Poly(dipiperidine-co-piperidine-co-2,3-Butanedione)

One gram (1 g) of 4,4′-bispiperdine was dissolved in 20 mL of THF and 10 mL methanol. 639 mg of 2,3-butanedione and 127 mg of piperidine was added. The reaction mixture was stirred at ambient temperature for 2 hours. 6.3 g of sodium triacetoxyborohydride was added. The resulting reaction mixture was stirred at ambient temperature for 18 hours. . The solvent was evaporated and the residue was dissolved in 100 mL of water. The pH was adjusted to 14 with 4M sodium hydroxide, and a white precipitate formed. The reaction mixture was filtered and the residue was dissolved in 1.2M hydrochloric acid to a pH of 1. The solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-27

### Synthesis of Poly(trimethylene dipiperidine:piperidine-co-2,2-Dimethyl-3,5-Hexanedione)

One gram (1 g) of 4,4′-trimethylenedipiperdine was dissolved in 30 mL of THF. 845 mg of 2,2-dimethyl-3,5-hexanedione and 101 mg of piperidine was added. The reaction mixture was stirred at ambient temperature for 2 hours. 5.0 g of sodium triacetoxyborohydride was added. The resulting reaction mixture was stirred at ambient temperature for 18 hours. . The solvent was evaporated and the residue was dissolved in 25 mL of 1.2M hydrochloric acid. The aqueous phase was extracted with 100 mL methyl tent-butyl ether. The pH was adjusted to 14 with 4M sodium hydroxide and the water phase was extracted with 100 mL dichloromethane (DCM)(repeated three times). The DCM was dried, filtered and evaporated. The residue was dissolved in 100 mL 1.2M hydrochloric acid. The solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-28

### Synthesis of Poly(trimethylene dipiperidine:piperidine-co-(±)-Camphorquinone)

One gram (1 g) of 4,4′-trimethylenedipiperdine was dissolved in 30 mL of THF. 987 mg of (±)-camphorquinone and 101 mg of piperidine was added. The reaction mixture was stirred at ambient temperature for 2 hours. 5.0 g of sodium triacetoxyborohydride was added. The resulting reaction mixture was stirred at ambient temperature for 18 hours. . The solvent was evaporated and the residue was dissolved in 25 mL of 1.2M hydrochloric acid. The aqueous phase was extracted with 100 mL methyl tent-butyl ether. The pH was adjusted to 14 with 4M sodium hydroxide and the water phase was extracted with 100 mL dichloromethane (DCM) (repeated three times). The DCM was dried, filtered and evaporated. The residue was dissolved in 100 mL 1.2M hydrochloric acid and the solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-29

### Synthesis of Poly(5-((3-(4-(3-(1-(3-aminopropyl)piperidin-4-yl)propyl)piperidin-1-yl)propyl)amino)-5-oxopentanoic acid)

### Example 3-29(a)

### Synthesis of 3,3′-(propane-1,3-diylbis(piperidine-4,1-diyl))dipropanenitrile

Seven grams (7 g) of 4,4′-trimethylenedipiperdine was ground into a fine powder with mortar and pestle and suspended in 80 mL water. The slurry was heated to 40 ° C. and acrylonitrile was added dropwise to the reaction mixture. After completed addition the reaction was left for 1 hour and left to cool to room temperature. The product, which precipitates out, was filtered and washed with water followed by freezing and lyophilizing.

### Example 3-29(b)

### Synthesis of 3,3′-(propane-1,3-diylbis(piperidine-4,1-diyl))bis(propan-1-amine)

Four grams (4 g) of 3,3′-(propane-1,3-diylbis(piperidine-4,1-diyl))dipropanenitrile was dissolved in 75 mL methanol and 3 g Raney cobalt was added. The slurry was sealed in a Parr reactor and hydrogen was slowly bubbled through the solution for 5 minutes. The reactor was then pressurized to 30 bars and the reaction was left for 72 hours. The reactor was de-pressurized and the reaction slurry was passed through a pad of celite. The methanol was evaporated leaving pure product.

### Example 3-29(c)

### Synthesis of Poly(5-((3-(4-(3-(1-(3-aminopropyl)piperidin-4-yl)propyl)piperidin-1-yl)propyl)amino)-5-oxopentanoic acid)

714 mg of 3,3′-(propane-1,3-diylbis(piperidine-4,1-diyl))bis(propan-1-amine) was dissolved in 13 mL dimethylformamide and 597 mg bis(2,5-dioxopyrrolidin-1-yl) glutarate in 5 mL dimethylformamide was added. The reaction was left for 12 hours at 50 ° C. and was triturated with methyl tent-butyl ether after having cooled to ambient temperature. The polymer was filtered and dissolved in 30 mL 1.2M hydrochloric acid and the solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-30

### Synthesis of Poly(6-((3-(4-(3-(1-(3-aminopropyl)piperidin-4-yl)propyl)piperidin-1-yl)propyl)amino)-6-oxohexanoic acid)

389 mg of 3,3′-(propane-1,3-diylbis(piperidine-4,1-diyl))bis(propan-1-amine) and 340 mg bis(2,5-dioxopyrrolidin-1-yl) adipate was dissolved in 7 mL dimethylformamide. The reaction was left for 12 hours at 50° C. and was triturated with methyl tent-butyl ether after having cooled to ambient temperature. The polymer was filtered and dissolved in 20 mL 1.2M hydrochloric acid and the solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-31

### Synthesis of Poly(6-((3-(4-(3-(1-(3-aminopropyl)piperidin-4-yl)propyl)piperidin-1-yl)propyl)amino)-6-oxooctanoic acid)

185 mg of 3,3′-(propane-1,3-diylbis(piperidine-4,1-diyl))bis(propan-1-amine) was dissolved in 1.4 mL dimethylformamide and 174 mg bis(2,5-dioxopyrrolidin-1-yl) octanedioate in 2 mL dimethylformamide was added. The reaction was left for 12 hours at 50 ° C. and was triturated with methyl tent-butyl ether after having cooled to ambient temperature. The polymer was filtered and dissolved in 20 mL 1.2M hydrochloric acid and the solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-32

### Synthesis of Poly(1-(3-(4-(3-(1-(3-aminopropyl)piperidin-4-yl)propyl)piperidin-1-yl)propyl)-3-(4-ureidobutyl)urea)

500 mg of 3,3′-(propane-1,3-diylbis(piperidine-4,1-diyl))bis(propan-1-amine) was dissolved in 3 mL dimethylformamide together with 8.3 mg N,N-dimethylpyridin-4-amine and 159 mg 1,4-diisocyanatobutane in 3 mL dichloromethane was added. The reaction was left for 12 hours at 50 ° C. and was triturated with methyl tent-butyl ether after having cooled to ambient temperature. The polymer was filtered and dissolved in 30 mL 1.2M hydrochloric acid and the solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-33

### Synthesis of Poly(6-((3-(4-(3-aminopropyl) piperazin-1-yl)propyl)amino)-6-oxohexanoic acid)

250 mg of 3,3′-(piperazine-1,4-diyl)bis(propan-1-amine) and 343 mg bis(2,5-dioxopyrrolidin-1-yl) adipate was dissolved in 7 mL dimethylformamide. The reaction was left for 12 hours at 50 ° C. and was triturated with methyl tent-butyl ether after having cooled to ambient temperature. The polymer was filtered and dissolved in 20 mL 1.2M hydrochloric acid and the solution was passed through a 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-34

### Synthesis of Poly(1-(2-((6-aminohexyl)amino)-2-oxoethyl)-4-(carboxymethyl)-1,4-diazabicyclo [2.2.2]octane-1,4-diium bromide)

### Example 3-34

### (a): Synthesis of 1,4-bis(2-ethoxy-2-oxoethyl)-1,4-diazabicyclo[2.2.2]octane-1,4-diium bromide

One gram (1 g) 1,4-diazabicyclo[2.2.2]octane was dissolved in 7 mL acetonitrile. To this solution was added 1.5 g ethyl 2-bromoacetate dissolved in 7 mL acetonitrile. The reaction was continued for 12 hours after which the solvent was evaporated. The residue was taken up in a small volume ethyl acetate and the salt triturated by adding a large volume of diethyl ether. The salt was filtered and re-dissolved in 7 mL acetonitrile and again 1.5 g ethyl 2-bromoacetate dissolved in 7 mL acetonitrile was added. After 12 hours the reaction was diluted with acetonitrile and the precipitate was filtered and dried.

### Example 3-34(b)

### Synthesis of 1,4-bis(2-oxo-2-phenoxyethyl)-1,4-diazabicyclo[2.2.2]octane-1,4-diium bromide

One gram (1 g) 1,4-diazabicyclo[2.2.2]octane was dissolved in 7 mL acetonitrile. To this solution was added 5.7 g phenyl 2-bromoacetate dissolved in 7 mL acetonitrile. After one hour the reaction was diluted with acetonitrile to facilitate stirring due to the heavy precipitate. After 12 hours the reaction was further diluted with acetonitrile and the precipitate was filtered and dried.

### Example 3-34(c)

### Synthesis of Poly(1-(2-((6-aminohexyl)amino)-2-oxoethyl)-4-(carboxymethyl)-1,4-diazabicyclo[2.2.2]octane-1,4-diium bromide) (1:1 diester/diamine)

250 mg 1,4-bis(2-ethoxy-2-oxoethyl)-1,4-diazabicyclo[2.2.2]octane-1,4-diium bromide was mixed with 65 mg 1,6-diaminohexane and the reaction was heated at 100 ° C. for 24 hours. The reaction mixture was dissolved in a few milliliters water and transferred to 500Da molecular weight cut-off dialysis tubing. The reaction was dialyzed in 5L water over night; the water was changed to fresh 5L and then left over night. The aqueous solution was frozen and lyophilized until dry.

### Example 3-34(d)

### Synthesis of Poly(1-(2-((6-aminohexyl)amino)-2-oxoethyl)-4-(carboxymethyl)-1,4-diazabicyclo[2.2.2]octane-1,4-diium bromide) (1:1 diester/diamine) [Alternative route]

250 mg 1,4-bis(2-oxo-2-phenoxyethyl)-1,4-diazabicyclo[2.2.2]octane-1,4-diium bromide was mixed with 54 mg 1,6-diaminohexane and the reaction was heated at 100 ° C. for 24 hours. The reaction mixture was dissolved in a few milliliters water and transferred to 500 Da molecular weight cut-off dialysis tubing. The reaction was dialyzed in 5 L water over night; the water was changed to fresh 5 L and then left over night. The aqueous solution was frozen and lyophilized until dry.

### Example 3-34(e)

### Synthesis of Poly(1-(2-((6-aminohexyl)amino)-2-oxoethyl)-4-(carboxymethyl)-1,4-diazabicyclo[2.2.2]octane-1,4-diium bromide) (1:2 diester/diamine)

250 mg 1,4-bis(2-ethoxy-2-oxoethyl)-1,4-diazabicyclo[2.2.2]octane-1,4-diium bromide was mixed with 130 mg 1,6-diaminohexane and the reaction was heated at 100 ° C. for 24 hours. The reaction mixture was dissolved in a few milliliters water and transferred to 500Da molecular weight cut-off dialysis tubing. The reaction was dialyzed in 5L water over night; the water was changed to fresh 5L and then left over night. The aqueous solution was frozen and lyophilized until dry.

### Example 3-35

### Synthesis of Poly(1-(2-((8-aminooctyl)amino)-2-oxoethyl)-4-(carboxymethyl)-1,4-diazabicyclo [2.2.2]octane-1,4-diium bromide)

### Example 3-35(a)

### Synthesis of Poly(1-(2-((8-aminooctyl)amino)-2-oxoethyl)-4-(carboxymethyl)-1,4-diazabicyclo [2.2.2] octane-1,4-diium bromide) (1:1 diester/diamine)

250 mg 1,4-bis(2-oxo-2-phenoxyethyl)-1,4-diazabicyclo[2.2.2]octane-1,4-diium bromide was mixed with 81 mg 1,6-diaminohexane and the reaction was heated at 100 ° C. for 24 hours. The reaction mixture was dissolved in a few milliliters water and transferred to 500Da molecular weight cut-off dialysis tubing. The reaction was dialyzed in 5L water over night; the water was changed to fresh 5L and then left over night. The aqueous solution was frozen and lyophilized until dry.

### Example 3-35(b)

### Synthesis of Poly(1-(2-((8-aminooctyl)amino)-2-oxoethyl)-4-(carboxymethyl)-1,4-diazabicyclo [2.2.2]octane-1,4-diium bromide) (1:2 diester/diamine)

250 mg 1,4-bis(2-oxo-2-phenoxyethyl)-1,4-diazabicyclo[2.2.2]octane-1,4-diium bromide was mixed with 162 mg 1,6-diaminohexane and the reaction was heated at 100 ° C. for 24 hours. The reaction mixture was dissolved in a few milliliters water and transferred to 500Da molecular weight cut-off dialysis tubing. The reaction was dialyzed in 5L water over night; the water was changed to fresh 5L and then left over night. The aqueous solution was frozen and lyophilized until dry.

### Example 3-36

### Synthesis of Poly((4-((2-aminoethyl)thio)-6-((1-((3-aminopropyl) amino)-4-methyl-1-oxopentan-2-yl)amino)-1,3,5-triazin-2-yl)leucine)

### Example 3-36(a)

### Synthesis of dimethyl 2,2′4(6-chloro-1,3,5-triazine-2,4-diyl)bis(azanediyl))bis(4-methylpentanoate)

738 mg triazine and 6.7 g sodiumbicarbonate were slurried in 20 mL acetone and cooled in an ice-water bath. To this slurry was added a solution of 1.6 g L-Leucine methyl ester hydrochloride in a 20 mL acetone/20 mL water mixture. The reaction was taken out of the cooling bath and the reaction was left to go up to room temperature overnight. The reaction was made acidic with 4M hydrochloric acid and the acetone was evaporated. The pure product precipitates and is collected by filtration.

### Example 3-36(b)

### Synthesis of dimethyl 2,2′-((6-((2-((tert-butoxycarbonyl) amino)ethyl)thio)-1,3,5-triazine-2,4-diyl)bis(azanediyl))bis(4-methylpentanoate)

0.54 g dimethyl 2,2′-((6-chloro-1,3,5-triazine-2,4-diyl)bis(azanediyl))bis(4-methylpentanoate) and 0.35 g tent-butyl (2-mercaptoethyl)carbamate were dissolved in 4 mL dimethylformamide. 64 mg NaH as a 60 wt. % mull was added (OBS! hydrogen formation) and the reaction was aged for one hour. The reaction was diluted with methyl tent-butyl ether and the organics was extracted three times with brine (salts fall out the first time. These were re-dissolved by adding small portions of water). The ether was dried over MgSO₄ and evaporated. At first the crude was passed through a 50 g silica column using ethyl acetate/hexane (gradient from 5% ethyl acetate to 50% over 10 column volumes) and then through a 10 g column with dichloromethane/ethyl acetate (gradient from 0% ethyl acetate to 5% over 15 column volumes).

### Example 3-36(c)

### Synthesis of Poly((4-((2-aminoethyl)thio)-6-((1-((3-aminopropyl) amino)-4-methyl-1-oxopentan-2-yl)amino)-1,3,5-triazin-2-yl)leucine)

331 mg dimethyl 2,2′-((6-((2-((tert-butoxycarbonyl) amino)ethyl)thio)-1,3,5-triazine-2,4-diyl)bis(azanediyl))bis(4-methylpentanoate) was mixed with 66 mg propane-1,3-diamine and the reaction was heated at 110 ° C. for 24 hours. The gummy mass was dissolved in 2 mL dichloromethane and 2 mL trifluoroacetic acid and stirred for three hours before being evaporated to dryness. The residue was dissolved in 20 mL 1.2M hydrochloric acid and the solution was passed through a 1 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-37

### Synthesis of Poly((4-((2-aminoethyl)thio)-6-((2-((3-aminopropyl) amino)-2-oxoethyl)amino)-1,3,5-triazin-2-yl)glycine)

### Example 3-37(a)

### Synthesis of diethyl 2,2′4(6-chloro-1,3,5-triazine-2,4-diyl)bis(azanediyl))diacetate

1.4 g ethyl glycinate hydrochloride and 2.6 g di-iso-propylethylamine were slurried in 10 mL tetrahydrofuran (THF). 922 mg triazine was dissolved in 10 mL tetrahydrofuran and added to the reaction mixture. After 30 minutes 1 mL of water/acetone (1:1) was added, forming a two phase system that was stirred rapidly. The reaction was left over night and subsequently diluted with more THF before being extracted with salt water (not brine). The water phase was re-extracted with more THF and the combined organic phase was concentrated under vacuum until the product started falling out of solution. The product was forced out of solution by adding 75 mL water and filtered.

### Example 3-37(b)

### Synthesis of diethyl 2,2′-((6-((2-((tert-butoxycarbonyl)amino) ethypthio)-1,3,5-triazine-2,4-diyl)bis(azanediyl))diacetate

0.36 g tent-butyl (2-mercaptoethyl)carbamate was dissolved in 10 mL diethyl ether and 64 mg NaH was added. The suspension was stirred for 45 minutes and then the reaction mixture was centrifuged. The supernatant was discarded and the white powder re-suspended in diethyl ether. The washing procedure was repeated twice more before the white mass was transferred to a flask and the diethyl ether was evaporated. The sodium salt was dissolved in 4 mL dimethylformamide and 0.32 g diethyl 2,2′-((6-chloro-1,3,5-triazine-2,4-diyl)bis(azanediyl))diacetate was added. The reaction was left for one hour before being diluted with methyl tent-butyl ether and the organic phase was extracted three times with brine (salts fall out the first time. These were re-dissolved by adding small portions of water). The ether was dried over MgSO₄ and evaporated. At first the crude was passed through a 50 g silica column using ethyl acetate/hexane (gradient from 5% ethyl acetate to 50% over 10 column volumes) and then through a 10 g column with dichloromethane/ethyl acetate (gradient from 0% ethyl acetate to 5% over 15 column volumes).

### Example 3-37(c)

### Synthesis of Poly((4-((2-aminoethyl)thio)-6-((2-((3-aminopropyl) amino)-2-oxoethyl)amino)-1,3,5-triazin-2-yl)glycine)

429 mg 2,2′((6-((2-((tert-butoxycarbonyl)amino) ethyl)thio)-1,3,5-triazine-2,4-diyl)bis(azanediyl))diacetate was mixed with 102 mg propane-1,3-diamine and the reaction was heated at 110 ° C. for 24 hours. The gummy mass was dissolved in 2 mL dichloromethane and 2 mL trifluoroacetic acid and stirred for three hours before being evaporated to dryness. The residue was dissolved in 20 mL 1.2M hydrochloric acid and the solution was passed through a 1 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

### Example 3-38:

### Synthesis of Poly((4-((6-amino-1-((3-aminopropyl)amino)-1-oxohexan-2-yl)amino)-6-(isopentylthio)-1,3,5-triazin-2-yl)lysine)

### Example 3-38(a)

### Synthesis of dimethyl 2,2′-((6-chloro-1,3,5-triazine-2,4-diyl)bis(azanediyl))bis(6-((tert-butoxycarbonyl)amino)hexanoate)

1.0 g methyl N6-(tert-butoxycarbonyl)lysinate hydrochloride salt and 829 g di-iso-propylethylamine were slurried in 3 mL tetrahydrofuran (THF). 295 mg triazine was dissolved in 3 mL tetrahydrofuran and added to the reaction mixture. After 30 minutes 1 mL of water/acetone (1:1) was added, forming a two phase system that was stirred rapidly. The reaction was left over night and subsequently diluted with more THF before being extracted with salt water (not brine). The water phase was re-extracted with more THF and the combined organic phase was concentrated under vacuum until the product started falling out of solution. The product was forced out of solution by adding 75 mL water and filtered.

### Example 3-38(b)

### Synthesis of dimethyl 2,2′-((6-(isopentylthio)-1,3,5-triazine-2,4-diyl)bis(azanediyl))bis(6-((tert-butoxycarbonyl)amino)hexanoate)

0.26 g 3-methylbutane-1-thiol was dissolved in 10 mL diethyl ether and 55 mg NaH was added. The suspension was stirred for 45 minutes and then the reaction mixture was centrifuged. The supernatant was discarded and the white powder re-suspended in diethyl ether. The washing procedure was repeated twice more before the white mass was transferred to a flask and the diethyl ether was evaporated. The sodium salt was dissolved in 4 mL dimethylformamide and 0.79 g dimethyl 2,2′-((6-chloro-1,3,5-triazine-2,4-diyl)bis(azanediyl))bis(6-((tertbutoxycarbonyl)amino)hexanoate) was added. The reaction was left for one hour before being diluted with methyl tent-butyl ether and the organic phase was extracted three times with brine (salts fall out the first time. These were re-dissolved by adding small portions of water). The ether was dried over MgSO₄ and evaporated. At first the crude was passed through a 50 g silica column using ethyl acetate/hexane (gradient from 5% ethyl acetate to 50% over 10 column volumes) and then through a 10 g column with dichloromethane/ethyl acetate (gradient from 0% ethyl acetate to 5% over 15 column volumes).

### Example 3-38(c):

### Synthesis of Poly((4-((6-amino-1-((3-aminopropyl)amino)-1-oxohexan-2-yl)amino)-6-(isopentylthio)-1,3,5-triazin-2-yl)lysine)

427 mg 2,2′-((6-((2-((tert-butoxycarbonyl)amino) ethyl)thio)-1,3,5-triazine-2,4-diyl)bis(azanediyl))diacetate was mixed with 45 mg propane-1,3-diamine and the reaction was heated at 110 ° C. for 24 hours. The gummy mass was dissolved in 2 mL dichloromethane and 2 mL trifluoroacetic acid and stirred for three hours before being evaporated to dryness. The residue was dissolved in 20 mL 1.2M hydrochloric acid and the solution was passed through a 1 10 KDa Macrosep filtration device (Pall corp.) by centrifugation at 5,000 rpm for 30 minutes. The retained material was diluted with water and the centrifugation process was repeated four more times. The material that passed through the Macrosep filter membrane was further purified with a 3 KDa Macrosep and the entire centrifugation process was repeated. Finally, the filtrate was passed through a 1 KDa Macrosep as above, yielding fractions containing polymers of four different molecular weight ranges, >10 KDa, <10 KDa and >3 KDa, <3 KDa and >1 KDa, and <1 KDa. Each fraction was dried by lyophilization resulting in fluffy white solids.

1. A compound comprising the structure of Formula (I): 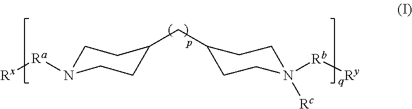
or a pharmaceutically acceptable salt thereof, wherein:
p is 0, 1, 2, 3, or 4
q is an integer from 2 to 10,000;
R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
R^{x} and R^{y} are each independently a pharmaceutically acceptable end group or are taken together with the carbons to which they are attached to form a 3 to 10 member ring,
wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide.

2. (canceled)

3. A compound comprising the structure of Formula (III): 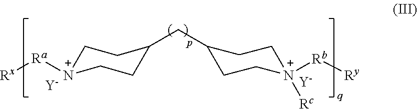
or a pharmaceutically acceptable salt thereof, wherein:
p is 0, 1, 2, 3, or 4
q is an integer from 2 to 10,000;
R^{a} and R^{b} are each independently absent or a substituted or unsubstituted group selected from (C₁-C₁₀)alkyl, (C₂-C₉)heteroalkyl, (C₃-C₁₀)cycloalkyl, (C₂-C₉)heterocycloalkyl, C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₃-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{x} and R^{y} are each independently a pharmaceutically acceptable end group or are taken together with the carbons to which they are attached to form a 3 to 10 member ring,
wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (X₁-C₁₀)alkyl-COOH, (C₃-C₁₀)cycloalkyl-COOH, —(O)CH₃, —OH, amide; and
Y⁻ is each independently a halo or any pharmaceutically acceptable anion.

4-5. (canceled)

52. A compound comprising the structure of Formula (IV): 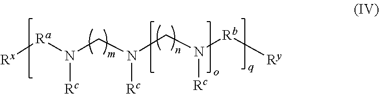
or a pharmaceutically acceptable salt thereof, wherein:
m is an integer from 0 to 15;
n is an integer from 0 to 15;
o is an integer from 0 to 10;
q is an integer from 2 to 10,000;
R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
R^{x} and R^{y} are each independently a pharmaceutically acceptable end group or are taken together with the carbons to which they are attached to form a 3 to 10 member ring,
wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide.

53. (canceled)

54. A compound comprising the structure of Formula (VI): 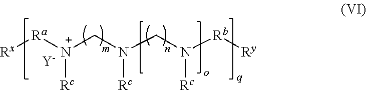
or a pharmaceutically acceptable salt thereof, wherein:
m is an integer from 0 to 15;
n is an integer from 0 to 15;
o is an integer from 0 to 10;
q is an integer from 2 to 10,000;
R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, -(O)CH₃, —OH, amide; and
R^{x} and R^{y} are each independently a pharmaceutically acceptable end group or are taken together with the carbons to which they are attached to form a 3 to 10 member ring,
wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
Y⁻ is a halo or any pharmaceutically acceptable anion.

55-57. (canceled)

58. A compound comprising the structure of Formula (X): 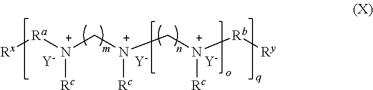
or a pharmaceutically acceptable salt thereof, wherein:
m is an integer from 0 to 15;
n is an integer from 0 to 15;
o is an integer from 0 to 10;
q is an integer from 2 to 10,000;
R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
R^{x} and R^{y} are each independently a pharmaceutically acceptable end group or are taken together with the carbons to which they are attached to form a 3 to 10 member ring,
wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
Y⁻ is each independently a halo or any pharmaceutically acceptable anion.

59. A compound comprising the structure of Formula (XI): 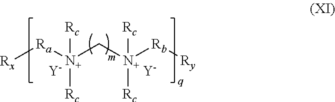
or a pharmaceutically acceptable salt thereof, wherein:
m is an integer from 0 to 15;
q is an integer from 2 to 10,000;
R^{a} and R^{b} are each independently absent or a substituted or unsubstituted group selected from substituted or unsubstituted (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{c} is each independently H or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
R^{x} and R^{y} are each independently a pharmaceutically acceptable end group or are taken together with the carbons to which they are attached to form a 3 to 10 member ring,
wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide; and
Y⁻ is each independently a halo or any pharmaceutically acceptable anion.

60-191. (canceled)

192. A pharmaceutical composition comprising the compound of claim 1.

193. A pharmaceutical composition comprising the compound of claim 3.

194. A pharmaceutical composition comprising the compound of claim 1 for use in the treatment or prevention of a condition selected from mucositis, oral mucositis, and infection.

195-214. (canceled)

215. A pharmaceutical composition comprising the compound of claim 3 for use in the treatment or prevention of a condition selected from mucositis, oral mucositis, and infection.

216. The pharmaceutical composition according to claim 9 for use in the treatment or prevention of a condition selected from mucositis, oral mucositis, and infection.

217. The pharmaceutical composition according to claim 10 for use in the treatment or prevention of a condition selected from mucositis, oral mucositis, and infection.

218-224. (canceled)

225. A method of treating a condition selected from mucositis, oral mucositis, and infection comprising administering a compound of claims 1.

226-229 (canceled)

230. A method of treating a condition selected from mucositis, oral mucositis, and infection, comprising administering a compound of claim 3.

231. The method according to claim 13, wherein the infection is a surgical site infection or a lung infection associated with cystic fibrosis.

232. The method according to claim 14, wherein the infection is a surgical site infection or a lung infection associated with cystic fibrosis.

233-279. (canceled)

280. A compound comprising the structure of Formula (XII): 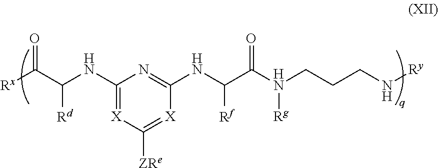
or a pharmaceutically acceptable salt thereof, wherein:
q is an integer from 2 to 10,000;
X is each independently N or P;
Z is NH, O, or S;
R^{d} and R^{e} are each independently H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide;
R^{f} and R^{g} are each independently H, or a substituted or unsubstituted group selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide, or R^{f} and R^{g} are taken together with the atoms to which they are attached to form a 4 to 10 member ring,
wherein the 4 to 10 member ring is optionally substituted by one to three groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, (C₁-C₁₀)alkyl-C(O)O—, COOH-(C₁-C₁₀)alkyl, COOH-(C₃-C₁₀)cycloalkyl, (C₁-C₁₀)alkyl-O—, —OH, —NH₂;
R^{x} and R^{y} are each independently a pharmaceutically acceptable end group or are taken together with the carbons to which they are attached to form a 3 to 10 member ring,
wherein the 3 to 10 member ring is optionally attached to a polymer or substituted by one to four groups selected from (C₁-C₁₀)alkyl, (C₂-C₉)heteroalkyl, (C₃-C₁₀)cycloalkyl, (C₂-C₉)heterocycloalkyl, (C₆-C₁₄)aryl, (C₂-C₉)heteroaryl, (C₁-C₁₀)alkylamine, carbonyl, —O(O)C—(C₁-C₁₀)alkyl, (C₁-C₁₀)alkyl-COOH, (C₃-C₁₀)cycloalkyl-COOH, —(O)CH₃, —OH, amide.

281-291. (canceled)

292. A pharmaceutical composition comprising the compound according to claim 17.

293. The pharmaceutical composition according to claim 18 for use in the treatment or prevention of a condition selected from mucositis, oral mucositis, and infection.

294. (canceled)

295. A method of treating a condition selected from mucositis, oral mucositis, and infection, comprising administering a compound according to claim 17.

296. The method according to claim 20, wherein the infection is a surgical site infection or a lung infection associated with cystic fibrosis.

297. (canceled)

298. The method according to claim 21, wherein the infection is a Pseudomonas aeruginosa lung infection.

299. The method according to claim 22, wherein biofilms are present in the Pseudomonas aeruginosa lung infection.
